(19) United States
(12) Patent Application Publication
(10) Pub. No.: US 2006/0088823 A1
(43) Pub. Date: Apr. 27, 2006

(54) Microarray gene expression profiling in clear cell renal cell carcinoma : prognosis and drug target identification

(76) Inventors: Brian Haab, Jenison, MI (US); Daniel Rhodes, Jenison, MI (US); Bin Tean Teh, Ada, MI (US); Masayuki Takahashi, Kentwood, MI (US)

Correspondence Address: MCKENNA LONG & ALDRIDGE LLP, 1900 K STREET, NW, WASHINGTON, DC, 20006, US

(21) Appl. No.: 10/473,173

(22) PCT Filed: Mar. 29, 2002
(86) PCT No.: PCT/US02/09576

§ 371 Date: Jan. 11, 2005

Related U.S. Application Data
(60) Provisional application No. 60/279,411, filed on Mar. 29, 2001.

(51) Int. Cl. C12Q 1/68

(52) U.S. Cl. 435/6

(57) ABSTRACT

A nucleic acid probe or a novel set of such probes in a microarray is provided. The probe or probe set is useful in the prognosis of patients with clear cell renal cell carcinoma (CC-RCC), wherein aggressive and non-agressive CC-RCC tumor types are characterized by differential expression profiles of genes that hybridize with one or more of these probes. Microarrays and kits

### BACKGROUND OF THE INVENTION

1. Field of the Invention

The present invention in the field of molecular biology and medicine relates to gene expression profiling of certain types of cancer and use of the profiles for prognosis. Specifically, the differential expression of a limited set of genes permits prognosis of an aggressive form of clear cell renal cell carcinoma (CC-RCC). Other genes are up- or down-regulated in most cases of CC-RCC; these are used for early diagnosis and/or drug discovery.

2. Description of the Background Art

CC-RCC, the most common form of adult kidney cancer, is caused by neoplasia of proximal renal tubular epithelium. CC-RCC is a prime example of a clinically heterogeneous disease for which treatment options are largely ineffective for advanced stage tumors. The cancer is more common in men than women, especially men over 55 years of age. It affects approximately 3/10,000 people; 18,000 new cases arise in the U.S. annually, of which about 8,000 result in death; worldwide fatalities are estimated to exceed 100,000 in 2001. CC-RCC represents 2% of all malignancies and 2% of all cancer-related deaths. Approximately 30% of patients present with metastatic disease and life expectancies averaging only 9 months.

RCC, originally named hypernephroma, was found to originate in the proximal renal tubule (Oberling et al., Nature (1986) 186:402-403) leading to its renaming to renal cell adenocarcinoma or renal cell carcinoma. RCC has been subdivided into clear, papillary, granular, and mixed cell variants based on cytoplasmic features. But the prognosis of RCC is based on staging and histological grading rather than the above classification.

A subtype of renal neoplasia with granular cell features, renal oncocytoma, which had excellent prognosis is described by Klein et al., Cancer (1976) 38:909-914. Thoenes et al., Virchows Arch B Cell Pathol Incl Mol Patiol. (1985) 48:207-217, describe a subtype of RCC with clear cell features, closely resembling an experimental renal tumor in rats, naming it chromophobe renal cell carcinoma. Fleming et al., Histopathology (1986);10: 1131-1141 describe yet another renal tumor, originating from the collecting ducts, named collecting duct carcinoma. Overlap of granular and clear cell features among tumors with marked clinical, pathologic, and phenotypic differences promoted the need for a new classification. Thoenes et al. (Pathol Res Pract. (1986) 181:125-143) proposed a new classification for renal tumors of tubular epithelial origin (the “Mainz classification”) based on conventional histopathologic criteria that include all the new entities described above.

The Mainz classification is now widely accepted; cytogenetic studies have confirmed characteristic genetic alterations of each tumor type (Yoshida et al., Cancer Res (1986) 46:2139-2147; Kovacs et al., Proc Natl Acad Sci USA (1988) 85:1571-1575 and Histopathology (1993) 22:1-8; Walter et al. Cancer Genet Cytogenet. (1989); 43:15-34).

The term RCC embraces a group of renal cancers all of which are derived from the renal tubular epithelium but each with distinct clinical, pathologic, phenotypic, and genotypic features.

| | Relative |
|---|---|
| Tumor Type | Frequency |
| Renal Cell Carcinoma: | |
| Clear Cell | 70% |
| Chromophil (eosinophil, basophil) | 15% |
| Chromophobe (typical, eosinophil) | 5% |
| Collecting Duct Carcinoma | 2% |
| Renal Oncocytoma | 5% |

CC-RCC is the most common adult renal neoplasm (70%). The tumor can be 1 cm in diameter when discovered (usually incidentally), or as bulky as several kilograms. Most often it manifests with pain, as a palpable mass or with hematuria; a variety of paraneoplastic syndromes have been described. CC-RCC may first manifest with metastases after being clinically silent for years. The characteristic gross appearance of the tumor is solid, lobulated, and yellow, with variegation due to necrosis and hemorrhage. Tumor may be well circumscribed, or may invade the perirenal adipose tissue or the renal vein. Cystic degeneration is common, though some tumors are predominantly cystic (Hartman et al., Urology (1986) 28:145-153). Of the 70% of patients with initially non-metastatic disease, approximately 30% relapse after surgery and usually succumb (Levy et al., J. Urolog. 159:1163-1167 (1999); Ljungberg, B et al., BJU Intl. 84: 405-411 (1999)).

The most common and consistent genetic finding in CC-RCC has been chromosomal (3p) loss (Tajara et al., Cancer Genet Cytogenet (1988) 31:75-82), along with amutation in the von Hippel-Lindau (VHL) gene in the other chromosome 3. In about 50% of sporadic CC-RCC cases, the VHL gene, located in 3p25, was mutated (Gnarra J R et al., (1994) Nature Genet 7:85-90). Reports of frequent loss of heterozygosity (LOH) in chromosome 3p13 and 3p14 suggested that other CC-RCC related genes exist in this region. Indeed, there are families with familial CC-RCC not associated with the VHL gene or chromosome 3 translocations (Teh, B T et al., 1997, Lancet 349:848-849), further supporting the notion that other CC-RCC genes exist.

To date, there have been no effective tools to identify those patients who will go on to relapse. Though the stimulus for RCC neoplastic transformation has not been identified, many associations with etiologic factors have been evaluated. Cigarette smoking is a prime risk factor. Incidence of CC-RCC is significantly increased in endstage renal patients who develop acquired cystic kidney disease. Although the tumors typically arise in the renal cortex, they may invade the renal vein and extend into the inferior vena cava. Paraneoplastic syndromes such as hypercalcemia and hepatic dysfunction in the absence of liver metastases have been reported.

The Union Intemationale Contre le Cancer (UICC) recently developed an improved system for classifying CC-RCC known as the “TNM” classification (referring to tumor, lymph node and metastasis). T, N, and M categories are determined by physical examination and imaging. (Sobin, L. H. et al., eds., TNM classification of malignant tumors. 5th ed. (John Wiley & Sons, New York 1997). This system is set forth in the table below.

Approximately one-third of initially diagnosed CC-RCC patients present with metastatic disease, and 40% of individuals undergoing surgical resection or radical nephrectomy will eventually develop metastasis. Among individuals with metastatic disease, approximately 75% exhibit lung metastasis, 36% have lymph node and/or soft tissue involvement, 20% have bone involvement, and 18% have liver involvement. The literature also reports low incidences of metastasis in contralateral adrenal glands, brain, uvula, diaphragm, and digits (Levy et al., supra). Spontaneous regression of metastases after nephrectomy occurs primarily in men with pulmonary. metastasis and are not equated with long-term cure. The frequency of spontaneous regression is only 0.4% and may reflect the development and/or enhancement of immune responses.

### TNM Clinical Classification

T—Primary Tumor

TX Primary tumor cannot be assessed

T0 No evidence of primary tumor

T1 Tumor is ≦7.0 cm in greatest dimension, limited to the kidney

T2 Tumor is >7.0 cm in greatest dimension, limited to the kidney

T3 Tumor extends into major veins or invades adrenal or perinephric tissues but not beyond Gerota fascia
T3a Tumor invades adrenal gland or perinephric tissues but not beyond Gerota fascia
T3b Tumor grossly extends into renal vein(s) or vena cava below diaphragm
T3c Tumor grossly extends into vena cava above diaphragm

T4 Tumor invades beyond Gerota fascia

N—Regional Lymph Nodes (Hilar, Abdominal Para-Aortic, and Paracaval)

NX Regional lymph nodes cannot be assessed

N0 No regional lymph node metastasis

N1 Metastasis in a single regional lymph node

N2 Metastasis in more than one regional lymph node

M—Distant Metastasis

MX Distant metastasis cannot be assessed

M0 No distant metastasis

M1 Distant metastasis present

pTNM Pathological Classification: corresponds to the T, N, and M categories.

G-Histopathologic Grading

GX Grade of differentiation cannot be assessed

G1 Well differentiated

G2 Moderately differentiated

G3, 4 Poorly differentiated/undifferentiated

Stage Grouping

| | M | N | M |
|---|---|---|---|
| Stage I | T1 | N0 | M0 |
| Stage II | T2 | N0 | M0 |
| Stage III | T1 | N1 | M0 |
| | T2 | N1 | M0 |
| | T3 | N0, N1 | M0 |
| Stage N | T4 | N0, N1 | M0 |
| | Any T | N2 | M0 |
| | Any T | Any N | M1 |

Conventional treatment of primary CC-RCC is surgical excision. However, metastasis limits long term survival. In patients with symptomatically advanced CC-RCC, palliative nephrectomy and other tumor excisions may be the only therapeutic option (Ljungberg et al., supra). Radiotherapy appears to have only limited palliative effects, as CC-RCC's appear to be relatively radio-resistant. Chemotherapy, usually with vinblastine, hydroxyurea and/or BCNU, also shows limited efficacy and response rates to prolonged infusion of 5-fluorouracil range from <10% to 20% decrease in tumor size. (Dutcher et al., Proc Annu Meet Am Soc Clin Oncol (1996) 15:A725). Hormonal therapy has also yielded disappointing results (Bukowsli, Cancer (1997) 80:1198-1220).

Immunotherapy with cytokines such as interferons and interleukin-2 (Proleukin® from Chiron), and combinations of these agents is considered an encouraging area of therapeutic development.

The making of the present invention has focused the inventors' attention on the fact that CC-RCC may exist as two distinct types: aggressive and non-aggressive, and that this distinction is of prime clinical importance. In the aggressive form, the primary tumor grows more rapidly, tends to metastasize sooner, the metastases grow more rapidly, and patients die sooner. Patients manifesting the aggressive type typically manifest stages III or IV. Non-aggressive RCC, patients typically manifest at stages I or II.

Current diagnosis of CC-RCC is limited to histologic analysis (in addition to corporal imaging, e.g., by ultrasonography, CT scans and X-rays). However, these modalities lack the rigor to distinguish fully between aggressive and non-aggressive tumor phenotype as conceived by the present inventors. Moreover, delays in staging and diagnosis of primary tumors in pre-symptomatic patients narrows the window for successful treatment, particularly of aggressive tumors which may have progressed to metastatic tumor before initial diagnosis.

The marked heterogeneity of CC-RCC provides one of the greatest challenges in diagnosis and treatment. This complicates prognosis and hinders selection of the most appropriate therapy. With the publication of the sequence of the human genome and the advent of high-throughput genomic and proteomic screening technologies, the molecular classification of human cancers are beginning to improve and will surely lead to better diagnosis and more specifically tailored and effective treatment strategies.

Because approximately 30% of CC-RCC patients present with metastatic disease and a short life expectancy (see above) and, of those with initially non-metastatic disease, approximately 30% relapse after surgery, there is an urgent need in the art to identify this latter group of patients before relapse so that appropriate therapies can be offered. To date, no such prognostic tool exists. The present invention provides such a tool for the first time, supplementing the available diagnostic approaches with a genetic screening approach that distinguishes between aggressive and non-aggressive tumor types by the differential expression of certain selected genes, expressed sequence tags (ESTs), gene fragments, mRNAs, and other polynucleotides as described herein.

The present inventors and others (Golub, I R et al., (1999) Science 286:531-537; Alizadeh, A A et al. (2000) Nature 403:503-511; Perou, C M et al., (2000) Nature 406:747-752; Bittner, M et al., (2000) Nature 406:536-540) have proposed that gene expression profiling using microarray technology can uncover the underlying molecular heterogeneity of cancers, thus identifying new classification schemes and means for more accurate diagnosis and prognosis. Lander's group successfully distinguished between acute myeloid leukemia and acute lymphoblastic leukemia by gene expression profiles (Golub et al., supra). Alizadeh et al. (supra) identified two distinct forms of diffuse large B-cell lymphoma with significantly different prognoses. In these studies, the ability to arrive at a clinically relevant molecular distinction was dependent on known cellular or molecular differences which correlated with gene expression profiles. However, one cannot create a meaningful molecular classification of diseases for which such cellular/molecular information is unavailable. Moreover, many microarray-based gene expression studies have been limited to comparisons of malignant tissue with normal tissue (or related cell lines). Without follow-up clinical data, the most important molecular profiles and relationships may remain obscure. Genetic aberrations common to all CC-RCC (discovered by the present inventors and described herein) may be initial contributing factors to disease. However, the presently disclosed set of differentially expressed DNAs may be responsible for the ultimate course of the disease.

Relevant Genetic Markers

This section provides general information about a number of genes that the present inventors have found to be differentially expressed in CC-RCC of different clinical severity.

The gene for transforming growth factor βII (TGFβII) receptor (TGFβIIR) is of particular interest to this invention as the present inventors have discovered its down-regulation to be associated with aggressive CC-RCC. The activated TGFβIIR is a heteromeric complex transmembrane protein with intrinsic cytoplasmic serine-threonine kinase domains through which the receptor complex suppresses cellular proliferation via initiation of a tumor suppression pathway. The ligand for this receptor, TGFβ, has three known isoforms in mammals: TGFβ1, TGFβ2, and TGFβ3. These proteins are members of a ligand family for TGFβIIR (which includes activin and bone morphogenic protein).

TGFβs interact with the TGFβIIR which, in turn, recruits the complex formed between TGFβIR and ALK5 to form a heterotetrameric complex. This constitutively activates the TGFβIIR kinase (Markowitz et al., Cytokine Growth Factor Rev. (1996) 7:93-102). Other members of the TGFβ superfamily interact with different combinations of homologous type I and type II receptor serine-threonine kinases. The activated kinase phosphorylates TGFβIR at the GS box, a conserved sequence of Gly and Ser residues N-terminal to the kinase domain. A strong correlation exists between malignant progression and loss of sensitivity to the anti-proliferative effects of TGFβ, which is frequently associated with reduced expression or inactivation of TGFβ receptors (Kim et al., Cytokine Growth Factor Rev (2000) 11:159-168).

A number of mutations inactivate TGFβIIR They include truncation at amino acid 97, BAT-RII mutations (big polyadenine tract mutation in exon 3 of TGFβIR gene), Glu¹⁴² to stop, and single amino acid substitutions at various positions. BAT-RII is associated with frameshift mutation in a 10-bp polyadenine tract resulting in a truncated receptor that lacks the serine-threonine linase domain (Markowitz S et al., Science (1995) 268:1336-1338). Receptor mutations, like Thr³¹⁵ to Met, do not interfere with the kinase activity but nevertheless enhance metastatic potential by specifically impeding TGFβ-mediated growth arrest without affecting the induction of extracellular matrix formation (Grady W M et al., (1999) Cancer Res 59:320-346).

Whereas TGFβR mutations (other than the BAT-RII frameshift) are rare events in tumorigenesis, repression of TGF-βR expression appears to be a common mechanism enabling tumor cells to escape from negative growth regulation by TGFβ. Mutations inactivating TGFβIIR kinase prevent phosphorylation of Smad family proteins which participate in the tumor suppression pathway. However, a reduction in TGFβIIR signaling in tumor cells is often accompanied by increased expression and secretion of TGFβ which functions independently through its effects on tumor cells and promotes tumorigenesis and metastasis (Abou-Shady et al., (1999) Am. J Surg. 177:209-215).

Captopril an inhibitor of angiotensin converting enzyme (ACE) was shown to attenuate growth human CC-RCC xenografts in immunosuppressed mice (Hii, S I et al., (1998) Br J Cancer 77:880-883). Though captopril's action and role in tumor suppression is not understood, this molecule is known to up-regulate TGFβIIR expression indirectly (Miyakima A. et al., (2001) J Urol 165:616-620) and to be anti-angiogenic (Volpert O V et al., (1996) J Clin Invest 98: 671-679).

Tissue inhibitor of metalloproteinase 3 (TIMP3) is also of interest to the present invention as disclosed below and has been implicated in RCC in previous studies (Kugler, A. Anticancer Res. (1999) 19:1589-1592, Kugler, A., et al., (1996) J. Urol. 160:1914-1918; Lien, M., et al., (2000) Int. J. Cancer 85:801-804). Matrix metalloproteinases [E's) are a group of zinc dependant enzymes responsible for extracellular matrix (ECM) degradation. They include type IV collagenases and 92 kDa gelatinase (MMP-9). The balance between MMP and available free TIMP (TIMP3 is of interest to the present invention) determines the net MMP activity. The ECM serves as a barrier between endothelial cells and the underlying stroma. Metastatic cancer cells repeatedly cross this barrier in a process requiring proteolysis. Metastasis occurs when the MMP:TIMP ratio exceeds 1 (Kugler, A. supra). Conversely, down-regulation or an inactivating mutation in TIMP can also give rise to tumor progression and metastasis.

Kininogens and their cleavage products are conserved multifunctional proteins (Cottrell Ga., et al., (1966) Nature, 212: 838-839, Rawlings N D et al., (1990) J Mol. Evol. 30: 60-71). In humans, low molecular weight kininogen (LK, ˜65 kDa) and high molecular weight kininogen (HK, ˜120 kDa) are single chain glycoproteins made up of kinin domains. Specific hydrolysis by tissue and plasma kallikreins releases Lys-bradykinin (Lys-BK) and bradyknin (BK), respectively, and cleaves each HK into two disulfide-linked fragments (heavy and light chains). Both LK and HK result from alternative splicing of mRNA transcribed from a single 11 exon gene that maps to chromosome 3q26-qter in humans (Fong D et al., (1991) Human Genetics 87:189-192, Takagaki Y et al., (1985) J Biol. Chen:. 260:8601-8609). The importance of HK/LK and kinins to normal biologic function is supported by the fact that kininogens and kinins have been conserved through evolution, participate in multiple biologic processes including inflammation, regulation of blood pressure and vascular permeability, cardioprotection and pain modulation (Rocha et al., (1949) Amer J Physiol 156: 261-273), and by the ubiquity of kinin receptors in mammalian tissues.

### SUMMARY OF THE INVENTION

The present inventors set out to characterize CC-RCC at the molecular level by identifying genes whose expression was altered (up or down) in a large percentage of CC-RCC cases. Furthermore, using a clinically well-characterized patient population, they sought to correlate the global gene expression profiling of CC-RCC with tumor progression and clinical outcome, even in the absence of known cellular or molecular characteristics of these tumors.

They hypothesized that by correlating gene expression with clinical parameters, they would uncover a molecular classification scheme for CC-RCC and thus enhance the understanding of progression of this disease. In summary, the objectives were (1) to identify common features of renal cell tumorigenesis, specifically, genes that were regularly up- or down regulated; (2) to generate a molecular portrait of clinically heterogeneous CC-RCC; (3) to identify specific molecular signatures of CC-RCC associated with a particular clinical subset of tumors; and finally, (4) to assess the clinical utility of a particular set of genes as a prognostic tool.

Beyond prognosis and defining new sub-types of disease, the discovery of a set of differentially expressed genes provides a basis for explaining the differences in aggressiveness and clinical outcome. Because genes that best discriminate two phenotypes are expected to be factors in that difference, the clinical follow-up data described herein allows investigation of genes with expression profiles unique to a particular clinical subtype.

Finally, use of the methods and compositions described herein permit identification of (A) proteins whose detection provide an early diagnostic approach to CC-RCC proteins as well (3) drug targets as the products of genes (i) whose expression is commonly altered in CC-RCC or (ii) whose activity is altered in a disease phenotype-selective manner. Thus, by discovering that a particular gene is differentially regulated in aggressive CC-RCC, one can focus on developing drugs that (1) correct down regulation or suppress up-regulation, for example by acting on cellular pathways that stimulate expression of this gene, (2) act directly on the protein product, or (c) bypass the step in a cellular pathway mediated by the product of this gene.

The present inventors have discovered a set of expressed nucleic acid markers through statistical clustering analysis, whose differential expression is indicative of heterogeneous CC-RCC disease manifestation.

The present invention provides a nucleic acid probe or a set of probes (preferably between 2 and 217 in number) and a microarray comprising these DNA markers as probes for the gene expression levels that are characteristic of CC-RCC tumor tissue compared to normal tissue from the same kidney. In one embodiment, the presence and levels of mRNA in a tissue being analyzed are screened using methods known in the art (i.e., Southern/Northern/Western blotting, gel electrophoresis, RFLP, SSCP). The invention is further directed to a method of implementing the microarray technology for disease prognosis (aggressive vs. non-aggressive CC-RCC) thereby supplementing currently available prognostic techniques (radiologic imaging) and pathological classification.

Use of the accurate, objective molecular methods described herein will inform physicians about which patients require heightened observation and additional, e.g., adjuvant, therapies—for example patients presenting with low stage CC-RCCs that appear on their face to be non-aggressive by conventional criteria, but that have the aggressive type molecular signatures as described herein. Moreover, in the case of patients presenting with higher stage CC-RCCs that might mistakenly be diagnosed as aggressive, but which have the non-aggressive molecular signature, this invention facilitates withholding of unnecessarily aggressive treatment while maintaining appropriate vigilance.

Thus, the present invention is directed to a prognostic microarray composition of at least one oligonucleotide or polynucleotide probe from a set of probes immobilized to a solid surface in a predetermined order such that a row of pixels corresponds to replicates of one distinct probe from the set. The probes are complementary to nucleic acid sequences expressed differentially in aggressive as compared to non-aggressive types of CC-RCC. The probes are preferably any of SEQ ID NO:1-SEQ ID NO:39 inclusive, SEQ ID NO:139 or SEQ ID NO:332-SEQ ID NO:497, inclusive. The nucleic acid sequences hybridize to the probes under high stringency conditions.

The microarray may comprise at least about 10 probes, or in another embodiment, at least about 39 or even at least about 206 probes, which probes are complementary to nucleic acid sequences expressed differentially in aggressive as compared to non-aggressive types of CC-RCC. These probes are preferably at least about 15 nucleotides in length.

The microarray of the present invention can be used to assay expressed nucleic acid samples (representing genes differentially expressed in normal kidney versus CC-RCC tumor tissue) for one or more individual subject's tumor or normal tissue, wherein each sample from an individual subject's tumor or normal tissue is spotted column-wise on the pixels of the microarray probes. The microarray can comprise at least 10, or, in another embodiment, at least about 99, or at least about 291 probes.

In one embodiment, the composition comprises the microarray to which are hybridized and thus immobilized, expressed nucleic acids from the subject. Preferably, hybridization is performed under stringent conditions.

The above microarray probes can comprise nucleotides having at least one modified phosphate backbone, e.g., phosphorothioate, a phosphoridothioate, a phosphoramidothioate, a phosphoramidate, a phosphordiimidate, a methylsphosphonate, an alkyl phosphotriester, 3′-aminopropyl, a formacetal, or analogues thereof.

Also provided is a composition comprising a set of two or more oligonucleotide or polynucleotide probes, each of which hybridizes with part or all of a coding sequence that is differentially expressed in aggressive type CC-RCC compared to non-aggressive type CC-RCC. The above set of probes can comprise at least about 10 probes, or, in another embodiment, at least about 39 probes, or even at least about 206 probes.

The differentially expressed nucleic acid sequences detected by the probes may be ones that are up-regulated or down-regulation in one form of CC-RCC compared to normal tissue or compared to the other form of CC-RCC (aggressive vs. non-aggressive).

The above probes are typically of mammalian, preferably human, origin.

Also provided is a method of predicting whether a subject with a CC-RCC has non-aggressive or aggressive-type CC-RCC. In this method, the expression of nucleic acids from the subject's normal kidney tissue versus kidney tumor tissue is compared in its hybridization, preferably at high stringency conditions, with one or more oligonucleotide or polynucleotide probes as above, preferably probes selected from those having the sequence SEQ ID NO:1-SEQ ID NO:21 or SEQ ID NO:22-SEQ ID NO:39.

In one embodiment using probes of the sequence SEQ ID NO:1-SEQ ID NO:21, up-regulation of at least 2-fold, preferably 3-fold, more preferably 4-fold, in tumor tissue is indicative of non-aggressive CC-RCC.

In another embodiment using probes of the sequence SEQ ID NO:22-SEQ ID NO:39, down-regulation of at least 2-fold, preferably 3-fold, more preferably 4-fold, in tumor tissue is indicative of aggressive CC-RCC.

In the above methods, the nucleic acids from the tumor and the tissue are detectably labeled, preferably with a fluorescent label prior to the hybridization. With fluorescent labels, hybridization is detected as a fluorescent signal bound to the probe.

In one embodiment of the above method, the probes are immobilized to a solid surface of a microarray as pixels arranged in rows, and the expressed nucleic acids from the tumor tissue or normal tissue samples are spotted column-wise onto the probe pixels.

Also provided is a method for the early diagnosis of a CC-RCC tumor in a subject prior to physical or radiological evidence of the tumor. In this method a protein product of at least one gene is selected based on its expression being up-regulated in a majority of CC-RCC patients. This protein product is preferably a secreted protein or a cell surface protein expressed in tissue readily accessible for assay. The presence or quantity of the protein product in a body fluid or a tissue or cell sample from the subject is determined. An increased level of the protein product compared to the level in a normal subject's fluid, tissue or cells (or another reference normal value) is indicative of the presence of a CC-RCC tumor in the subject.

This invention also provides is a method for diagnosing the recurrence of a CC-RCC tumor in a subject in whom a CC-RCC primary tumor has been excised or otherwise treated. In this method a protein product of at least one gene is selected based on its expression being up-regulated in a majority of CC-RCC patients. This protein product is preferably a secreted protein or a cell surface protein expressed in tissue readily accessible for assay. The presence or quantity of the protein product protein product in a body fluid or a tissue or cell sample from the subject is determined. An increase in the level of the protein product compared to the level in a normal subject's fluid, tissue or cells (or another reference normal value) is indicative of the presence of a recurrent CC-RCC tumor in the subject.

In both methods of early diagnosis and diagnosis of recurrence, the gene is preferably one that hybridizes with any one or more of SEQ ID NO:40-SEQ ID NO:68 or SEQ ID NO:140-SEQ ID NO:230, more preferably with one or more of SEQ ID NO:40-SEQ ID NO:68.

The invention also provides a kit comprising a microarray, reagents that facilitate hybridization of differentially expressed nucleic acid to the immobilized probes on the microarray, and a computer readable storage medium comprising logic which enables a processor to read data representing detection of hybridization. These kits are useful for the diagnosis of aggressive or non-aggressive CC-RCC.

In one embodiment of the provided kit, the reagents facilitate detection of fluorescence as the means for determining hybridization.

Also included is a kit comprising (a) the microarray or composition of any of claims 1-22; (b) means for carrying out hybridization of the nucleic acid to the probes; and (c) means for reading hybridization data. The hybridization data is preferably in the form of fluorescence data. The probes are preferably immobilized to the microarray.

### BRIEF DESCRIPTION OF THE DRAWINGS

FIG. 1 shows an unsupervised two-way clustering matrix for all 3,184 genes tested. Colored bars on the right represent nodes with high predictive scores. Patient groups appear at the top coded in red, blue or black: Red—poor outcomes; Blue—good outcome; Black—short follow-up period.

FIGS. 2A and 2B show a supervised two-way re-clustering matrix (FIG. 2A) of independent ‘predictive’ node 1281, and its respective dendrogram (FIG. 2B) displaying the similarity of patient samples based on a specific subsets of genes. Color code for patients as for FIG. 1. The colors appearing in the multicolor bar beneath the dendrogram appearing at the very bottom of FIG. 2B represent the average expression values for the subsets of genes for each patient.

FIGS. 3A and 3B show a supervised two-way re-clustering matrix (FIG. 3A) of independent ‘predictive’ node 3014, and its respective dendrogram (FIG. 3B) displaying the similarity of patient samples based on a specific subsets of genes. Color code for patients as for FIG. 1. The colors appearing in the multicolor bar beneath the dendrogram appearing at the very bottom of FIG. 3B represent the average expression values for the subsets of genes for each patient.

FIGS. 4A and 4B show a supervised two-way re-clustering matrix (FIG. 4A) of independent ‘predictive’ node 2199, and its respective dendrogram (FIG. 4B) displaying the similarity of patient samples based on a specific subsets of genes. Color code for patients as for FIG. 1. The colors appearing in the multicolor bar beneath the dendrogram appearing at the very bottom of FIG. 4B represent the average expression values for the subsets of genes for each patient.

FIG. 5 shows an expression matrix of a prognostic set of 51 genes (node 1281 from FIGS. 2A and 2B). Median centering of genes was not performed so that each square corresponds to the actual normalized gene expression level relative to normal tissue. The red bar labeled “A” marks genes mostly up regulated in low-risk, non-aggressive tumors. The green bar “B” marks genes mostly down regulated in high-risk, aggressive tumors.

FIG. 6 is shows clustering expression matrices of subsets of genes the expression of which was detected in 29 CC-RCC tumors. Rows represent individual polynucleotide probes (cDNAs or ESTs) immobilized to the slides; columns represent individual patient tumor samples (as fluorescently labeled cDNAs). Each square's color corresponds to the median-polished, normalized DNA expression value for a single gene in a single tumor relative to patient-matched normal renal tissue. Gene expression is either depicted in RED (above median), GREEN (below median), BLACK (equal to median) or GRAY (inadequate or missing data). The color saturation indicates the extent of divergence from the median. FIGS. 6A and 6B show supervised two-way re-clustering matrices of three independent ‘predictive’ nodes (reproduced as enlarged views in FIGS. 2A, 3A, and 4A). FIG. 6C shows the respective dendrograms displaying the similarity of patient samples based on specific subsets of genes (reproduced as enlarged views in FIGS. 2B, 3B, and 4B). Color code for patients: Red—poor outcomes; Blue—good outcome; Black—short follow-up period. The colors appearing in the multicolor bar beneath each dendrogram (FIG. 6C) represent the average expression values for the subsets of genes for each patient.

### DESCRIPTION OF THE PREFERRED EMBODIMENTS

In the present application, the terms “nucleic acid” and “polynucleotide” are used interchangeably and refer to both DNA and RNA (as well as peptide nucleic acids). The term “oligonucleotide” is not intended to be limited to a particular number of nucleotides and therefor overlaps with polynucleotide. Probes for gene expression analysis include those comprising ribonucleotides, deoxyribonucleotides, both or their analogues as described below. They may be poly- or oligonucleotides, without limitation of length. Preferred lengths are described below.

The present invention uses cDNA microarrays to probe for, and to determine the relative expression of, target genes of interest in a tissue sample of CC-RCC.

Microarrays are orderly arrangements of spatially resolved samples or probes (in the present invention cDNAs of known sequence ranging in size from 200 to 2000 nucleotides), that allow for massively parallel gene expression and gene discovery studies (Lockhart D J et al., Nature (2000) 405(6788):827-836). The probes are immobilized to a solid substrate and made available to hybridize with their complementary strands as is described in the preferred embodiments (Phimister, Nature Genetics (1999) 21(supp):1-60).

The underlying concept of the microarray depends on base-pairing (hybridization) between purine and pyrimidine bases following the rules of Watson-Crick base pairing. Microarray technology adds automation to the process of resolving nucleic acids of particular identity and sequence present in an analyte sample by labeling, preferably with fluorescent labels, and subsequent hybridization to their complements immobilized to a solid support in microarray format. Array experiments employ common solid supports such as glass slides, microplates or standard blotting membranes, and can be created by hand or by robotic deposition of samples. Arrays are generally described as macroarrays or microarrays. Macroarrays contain sample spots of about 300 μm diameter or larger and can be easily imaged by existing gel and blot scanners. Sample spot sizes in microarrays are typically <200 μm in diameter, and these arrays usually contains thousands of spots. Microarrays require specialized robotics and imaging equipment that generally are commercially available and well-known in the art. However, the materials for a particular application are not necessarily available in convenient in kit form. The present invention provides microarrays useful for analysis and prognosis of CC-RCC samples.

DNA microarrays (DNA “chips”) are fabricated by high-speed robotics, preferably on glass (though nylon and other plastic substrates are used). An experiment with a single DNA chip can provide simultaneous information on thousands of genes—a dramatic increase in throughput (Reichert et al. (2000) Anal. Chen.72:6025-6029) when compared to traditional methods.

Two DNA microarray formats are preferred.
Format I: a cDNA probe (500˜5,000 bases) is immobilized to a solid surface such as glass using robotic spotting and exposed to a set of targets either separately or in a mixture. This method, traditionally called “DNA microarray,” is considered to have been developed at Stanford University (Ekins, R et al., Trends in Biotech (1999) 17:217-218).
Format II: an array of probes that are “natural” oligo- or polynucleotides (oligomers of 20˜80 bases), oligonucleotide analogues e.g., with phosphorothioate, methylphosphonate, phosphoramidate, or 3′-aminopropyl backbones), or peptide-nucleic acids (PNA) Probes may be synthesized either in situ (on-chip) or by conventional synthesis followed by on-chip immobilization.

The array is (1) exposed to an analyte comprising a detectable labeled, preferably fluorescent, sample nucleic acid (typically DNA), (2) allowed to hybridize, and (3) the identity and/or abundance of complementary sequences is determined.

| 1. Probe (cDNA or | 2. Chip | 3. Target | | |
|---|---|---|---|---|
| oligonucleotide of | fabrication (putting | (detectably labeled | | |
| known identity) | probes on the chip) | sample) | 4. Assay | 5. Readout |
| Small oligos, cDNA, | Photolithography, | PolyA-mRNA | Hybridization, long, | Fluorescence, |
| chromosome | pipette, drop-touch, | extraction, RT-PCR, | short, ligase, base | radioactivity, |
| | piezoelectric (ink- | cDNA isolation, | addition, electric, MS, | etc. |
| | jet), electric | melting | electrophoresis, flow | |
| | | | cytometry, PCR-Direct, | |
| | | | TaqMan ®, etc. | |

For analysis of the target nucleic acid of primary tumor tissue, the preferred analyte of this invention is isolated from tissue biopsies before they are stored or from fresh-frozen tumor tissue of the primary tumor which may be stored and/or cultured in standard culture media. For expression studies, poly(A)-containing mRNA is isolated using commercially available kits, e.g., from Invitrogen, Oligotex, or Qiagen. The isolated mRNA is reverse transcribed into cDNA in the presence of a labeled nucleotides. Fluorescent cDNA is generally synthesized using reverse transcriptase (e.g., Superscript II reverse-transcription kit from GIBCO-BRL) and nucleotides to 20 which is conjugated a fluorescent label. A preferred fluorescent label is Cy5 conjugated to dUTP and/or dCTP (from Amersham).

The present invention utilizes immobilized cDNA probes of anywhere between about 15 bases up to a full length cDNA, e.g., about 2000 bases. Preferred probes have about 100 bases. Optimal hybridization conditions (i.e., temperature, pH, ion and salt concentrations, and incubation time) are dependent on the length of the shortest probes as the limiting step and can be adjusted in a continuous fashion by varying the above parameters as is conventional in the art.

Several probe sequences described herein are cDNAs complementary to genes or gene fragments; some are ESTs. Those skilled in the art will appreciate that the probe of choice for a particular gene can be the full length coding sequence or any fragment thereof having at least about 15 nucleotides. Thus, when the full length sequence is known, the practitioner can select any appropriate fragment of that sequence. When the original results are obtained using partial sequence information (e.g., an EST probe), and when the full length sequence of which that EST is a fragment becomes available (e.g., in a genome database), the skilled artisan can select a longer fragment than the initial EST, as long as the length is at least about 15 nucleotides.

The present invention includes microarrays comprising one or more nucleic acid probes having hybridizable fragments of any length (from about 15 bases to full coding sequence) for the genes whose expression is to be analyzed. For purposes of the analysis, the full length sequence must not necessarily be known, as those of skill in the art will know how to obtain the fall length sequences using the sequence of a given EST and known data mining, bioinformatic, and DNA sequencing methodologies without undue experimentation.

The polynucleotide or oligonucleotide probes of the present invention may be native DNA or RNA molecules or an analogues of DNA or RNA. The present invention is not limited to the use of any particular DNA or RNA analogue; rather any one is useful provided that it is capable of adequate hybridization to the complementary DNA (or mRNA) in a test sample, has adequate resistance to nucleases and stability in the hybridization protocols employed. DNA or RNA may be made more resistant to nuclease degradation in vivo by modifying internucleoside linkages (e.g., methylphosphonates or phosphorothioates) or by incorporating modified nucleosides (e.g., 2′-0-methylribose or 1′-α-anomers) as described below.

A poly- or oligonucleotide may comprise at least one modified base moiety, for example, 5-fluorouracil, 5-bromouracil, 5-chlorouracil, 5-iodouracil, hypoxanthine, xanthine, 4-acetylcytosine, 5-(carboxyhydroxyhnethyl)uracil, 5-carboxymethylaminomethyl-ω-thiouridine, 5-carboxymethyl-aminomethyl uracil, dihydrouracil, β-D-galactosylqueosine, inosine, N6-isopentenyladenine, 1-methylguanine, 3-methyl-cytosine, 5-methylcytosine, N6-adenine, 7-methylguanine, 5-methylaminomethyluracil, 5-methoxyamino-methyl-2-thiouracil, β-D-mannosylqueosine, 5-methoxy-carboxymethyluracil, 5-methoxyuracil-2-methylthio-N6-iso-pentenyladenine, uracil-5-oxyacetic acid, butoxosine, pseudouracil, queuosine, 2-thio-cytosine, 5-methyl-2-thiouracil, 2-thiouracil, 4-thiouracil, 5-methyluracil, uracil-5-oxyacetic acid methylester, uracil-t-oxyacetic acid, 5-methyl-2-thiouracil, 3(3-amino-3-N-2-carboxypropyl) uracil and 2,6-diaminopurine.

The poly- or oligonucleotide may comprise at least one modified sugar moiety including, but not limited, to arabinose, 2-fluoroarabinose, xylulose, and hexose.

In yet another embodiment, the poly- or oligonucleotide probe comprises a modified phosphate backbone synthesized from a nucleotide having, for example, one of the following structures: a phosphorothioate, a phosphoridothioate, a phosphoramidothioate, a phosphoramidate, a phosphordiimidate, a methylsphosphonate, an alkyl phosphotriester, 3′-aminopropyl and a formacetal or analog thereof.

In yet another embodiment, the poly- or oligonucleotide probe is an α-anomeric oligonucleotide which forms specific double-stranded hybrids with complementary RNA in which, contrary to the usual β-units, the strands run parallel to each other (Gautier et al., 1987, Nucl. Acids Res. 15:6625-6641).

An oligonucleotide may be conjugated to another molecule, e.g., a peptide, a hybridization triggered cross-linking agent, a hybridization-triggered cleavage agent, etc., all of which are well-known in the art.

Oligonucleotides of this invention may be synthesized by standard methods known in the art, e.g. by use of an automated DNA synthesizer (such as are commercially available from Biosearch, Applied Biosystems, etc.). As examples, phosphorothioate oligonucleotides may be synthesized by the method of Stein et al., (Nucl. Acids Res. (1998) 16:3209, methylphosphonate oligonucleotides can be prepared by use of controlled pore glass polymer supports (Sarin et al., Proc. Natl. Acad. Sci. U.S.A. (1988) 85:7448-7451), etc.

Detectable Labels for Oligo- or Polynucleotide Probes

Preferred detectable labels include a radionuclides, fluorescers, fluorogens, a chromophore, a chromogen, a phosphorescer, a chemiluminescer or a bioluminescer. Examples of fluorescers or fluorogens are i fluorescein, rhodamine, dansyl, phycoerythrin, phycocyanin, allophycocyanin, o-phthaldehyde, fluorescamine, a fluorescein derivative, Oregon Green, Rhodamine Green, Rhodol Green or Texas Red.

Common fluorescent labels include fluorescein, rhodamine, dansyl, phycoerythrin, phycocyanin, allophycocyanin, o-phthaldehyde and fluorescamine. Most preferred are the labels described in the Examples, below.

The fluorophore must be excited by light of a particular wavelength to fluoresce. See, for example, Haugland, Handbook of Fluorescent Probes and Research Chemicals, Sixth Ed., Molecular Probes, Eugene, Oreg., 1996).

Fluorescein, fluorescein derivatives and fluorescein-like molecules such as Oregon Green™ and its derivatives, Rhodamine Green™ and Rhodol Green™, are coupled to amine groups using the isothiocyanate, succinimidyl ester or dichlorotriazinyl-reactive groups. Similarly, fluorophores may also be coupled to thiols using maleimide, iodoacetamide, and aziridine-reactive groups. The long wavelength rhodamines, which are basically Rhodamine Green™ derivatives with substituents on the nitrogens, are among the most photostable fluorescent labeling reagents known. Their spectra are not affected by changes in pH between 4 and 10, an important advantage over the fluoresceins for many biological applications. This group includes the tetramethylrhodamines, X-rhodamines and Texas Red™ derivatives. Other preferred fluorophores are those which are excited by ultraviolet light. Examples include cascade blue, coumarin derivatives, naphthalenes (of which dansyl chloride is a member), pyrenes and pyridyloxazole derivatives.

The present invention serves as a basis for even broader implementation of microarrays and gene expression in deducing critical pathways implicated in cancer. In the case of CC-RCC, which is the focus of the present invention, a database of known patient genetic profiles can be used to categorize each new CC-RCC patient. The gene expression profile of the newly diagnosed CC-RCC patient is compared to the known CC-RCC molecular database of patients, such as that described herein based on 29 patients in whom complete clinical follow-up information is available. This database will grow with each patient who is subjected to the present analysis as soon as his clinical outcome information becomes available. If the newly diagnosed patient's gene expression profile most closely resembles the profile of aggressive CC-RCC, as described herein, that patient will be so classified and treated accordingly, i.e., with more aggressive measures. Correspondingly, if a newly diagnosed patient's profile is that of the non-aggressive type, he will be treated accordingly, e.g., with less aggressive measures and careful clinical follow-up.

Considering the low response rates of CC-RCC patients to current therapies such as with interferon-α and interleukin-2 infusion, the report that the apoptosis following induction of TIMP3 (Ahonen, et al. (1998) Cancer Res 58:2310-2315) coupled with the discovery here that TIMP3 is down-regulated in aggressive CC-RCC points to a new potential therapeutic strategy that may include gene therapy. The present approach permits the identification of one or more appropriate targets for such therapy.

Drug Discovery Based on Gene Expression Profiling

The molecular profiling information described herein is also harnessed for the purpose of discovering drugs that are selected for their ability to correct or bypass the molecular alterations or derangements that are characteristic of CC-RCC, particularly those that are associated with its aggressive form. A number of approaches are available.

In one embodiment, CC-RCC cell lines are prepared from tumors using standard methods and are profiled using the present methods. Preferred cell lines are those that maintain the expression profile of the primary tumor from which they were derived. One or several CC-RCC cells lines may be used as a “general” panel; alternatively or additionally, cell lines from individual patients may be prepared and used. These cell lines are used to screen compounds, preferably by high-throughput screening (HTS) methods, for their ability to alter the expression of selected genes. Typically, small molecule libraries available from various commercial sources are tested by HTS protocols.

The molecular alterations in the cell line cells can be measured at the mRNA level (gene expression) applying the methods disclosed in detail herein. Alternatively, one may assay the protein product(s) of the selected gene(s). Thus, in the case of secreted or cell-surface proteins, expression can be assessed using immunoassay or other immunological methods including enzyme immunoassays (EIA), radioimmunoassay (RIA), immunofluorescence microscopy or flow cytometry. EIAs are described in greater detail in several references (Butler, J E, In: Structure of Antigens, Vol. 1 (Van Regenmortel, M., CRC Press, Boca Raton 1992, pp. 209-259; Butler, J E, “ELISA,” In: van Oss, C. J. et al. (eds), Immunochemistry, Marcel Dekker, Inc., New York, 1994, pp. 759-803; Butler, J E (ed.), Inmmunochemistry of Solid-Phase Immunoassay, CRC Press, Boca Raton, 1991). RIAs are discussed in Kirkdam and Hunter (eds.), Radioimmune Assay Methods, E. & S. Livingstone, Edinburgh, 1970.

In another approach, antisense RNAs or DNAs that specifically inhibit the transcription and/or translation of the targeted genes can be screened for specificity and efficacy using the present methods. Antisense compositions would be particularly useful for treating tumors in which a particular gene is up-regulated (e.g., the genes in Tables 2 and 3).

Diagnostic Methods

The protein products of genes that are upregulated in most cases of CC-RCC (e.g. Tables 2 and 3) are targets for early diagnostic assays of CC-RCC if the proteins can be detected by some assay means, e.g., immunoassay, in some accessible body fluid or tissue. The most useful diagnostic targets are secreted proteins which reach a measurable level in a body fluid before the tumor presents by other criteria discussed in the Background section. Thus, a sample of a body fluid such as such as plasma, serum, urine, saliva, cerebrospinal fluid, etc., is obtained from the subject being screened. The sample is subject to any known assay for the protein analyte. Alternatively, cells expressing the protein on their surface may be obtained, e.g., blood cells, by simple, conventional means. If the protein is a receptor or other cell surface structure, it can be detected and quantified by well-known methods such as flow cytometry, immunofluorescence, immunocytochemistry or immunohistochemistry, and the like.

Preferably, an antibody or other protein or peptide ligand for the target protein to be detected is used. In another embodiment where the gene product is a receptor, a peptidic or small molecule ligand for the receptor may be used in known assays as the basis for detection and quantitation.

In vivo methods with appropriately labeled binding partners for the protein targets, preferably antibodies, may also be used for diagnosis and prognosis, for example to image occult metastatic foci or for other types of in situ evaluations. These methods utilize include various radiographic, scintigraphic and other imaging methods well-known in the art (MRI, PET, etc.).

Suitable detectable labels include radioactive, fluorescent, fluorogenic, chromogenic, or other chemical labels. Useful radiolabels, which are detected simply by gamma counter, scintillation counter or autoradiography include ³H, ¹²⁵I, ¹³¹I, ³⁵S and ¹⁴C.

Common fluorescent labels include fluorescein, rhodamine, dansyl, phycoerythrin, phycocyanin, allophycocyanin, o-phthaldehyde and fluorescamine. The fluorophore, such as the dansyl group, must be excited by light of a particular wavelength to fluoresce. See, Haugland, Handbook of Fluorescent Probes and Research Chemicals, Sixth Ed., Molecular Probes, Eugene, Oreg., 1996). Fluorescein, fluorescein derivatives and fluorescein-like molecules such as Oregon Green™ and its derivatives, Rhodamine Green™ and Rhodol Green™, are coupled to amine groups using the isothiocyanate, succinimidyl ester or dichlorotriazinyl-reactive groups. Fluorophores may also be coupled to thiols using maleimide, iodoacetamide, and aziridine-reactive groups. The long wavelength rhodamines include the tetramethylrhodamines, X-rhodamines and Texas Red™ derivatives. Other preferred fluorophores for derivatizing the protein binding partner are those which are excited by ultraviolet light. Examples include cascade blue, coumarin derivatives, naphthalenes (of which dansyl chloride is a member), pyrenes and pyridyloxazole derivatives.

The protein (antibody or other ligand) can also be labeled for detection using fluorescence-emitting metals such as ¹⁵²Eu, or others of the lanthanide series. These metals can be attached to the protein using metal chelating groups such as diethylenetriaminepentaacetic acid (DTPA) or ethylenediaminetetraacetic acid (EDTA).

For in vivo diagnosis, radionuclides may be bound to protein either directly or indirectly using a chelating agent such as DTPA and EDTA which is chemically conjugated, coupled or bound (which terms are used interchangeably) to the protein. The chemistry of chelation is well known in the art. The key limiting factor on the chemistry of coupling is that the antibody or ligand must retain its ability to bind the target protein. A number of references disclose methods and compositions for complexing metals to macromolecules including description of useful chelating agents. The metals are preferably detectable metal atoms, including radionuclides, and are complexed to proteins and other molecules. See, for example, U.S. Pat. No. 5,627,286, U.S. Pat. No. 5,618,513, U.S. Pat. No. 5,567,408, U.S. Pat. No. 5,443,816, U.S. Pat. No. 5,561,220, all of which are incorporated by reference herein.

Any radionuclide having diagnostic (or therapeutic value) can be used. In a preferred embodiment, the radionuclide is a y -emitting or P-emitting radionuclide, for example, one selected from the lanthanide or actinide series of the elements. Positron-emitting radionuclides, e.g. ⁶⁸Ga or ⁶⁴Cu, may also be used. Suitable γ-emitting radionuclides include those which are useful in diagnostic imaging applications. The gamma -emitting radionuclides preferably have a half-life of from 1 hour to 40 days, preferably from 12 hours to 3 days. Examples of suitable y-emitting radionuclides include ⁶⁷Ga, ¹¹¹In, ^{99m}Tc, ¹⁶⁹Yb and ¹⁸Re. Examples of preferred radionuclides (ordered by atomic number) are ⁶⁷Cu, ⁶⁷Ga, ⁶⁸Ga, ⁷²As, ⁸⁹Zr, ⁹⁰Y, ⁹⁷Ru, ⁹⁹Tc, ¹¹¹In, ¹²³I, ¹²⁵I, ¹³¹I, ¹⁶⁹Yb, ¹⁸⁶Re, and ²⁰¹Tl. Though limited work have been done with positron-emitting radiometals as labels, certain proteins, such as transferrin and human serum albumin, have been labeled with ⁶⁸Ga,

A number of metals (not radioisotopes) useful for MRI include gadolinium, manganese, copper, iron, gold and europium. Gadolinium is most preferred. Dosage can vary from 0.01 mg/kg to 100 mg/kg.

In situ detection of the labeled protein may be accomplished by removing a histological specimen from a subject and examining it by microscopy under appropriate conditions to detect the label. Those of ordinary skill will readily perceive that any of a wide variety of histological methods (such as staining procedures) can be modified in order to achieve such in situ detection.

The compositions of the present invention may be used in diagnostic, prognostic or research procedures in conjunction with any appropriate cell, tissue, organ or biological sample of the desired animal species. By the term “biological sample” is intended any fluid or other material derived from the body of a normal or diseased subject, such as blood, serum, plasma, lymph, urine, saliva, tears, cerebrospinal fluid, milk, amniotic fluid, bile, ascites fluid, pus and the like. Also included within the meaning of this term is a organ or tissue extract and a culture fluid in which any cells or tissue preparation from the subject has been incubated.

An alternative diagnostic approach utilizes cDNA probes that are complementary to and thereby detect cells in which a gene associated with CC-RCC is upregulated by in situ hybridization with mRNA in these cells. The present invention provides methods for localizing target mRNA in cells using fluorescent in situ hybridization (FISH) with labeled cDNA probes having a sequence that hybridizes with the mRNA of an upregulated gene. The basic principle of FISH is that DNA or RNA in the prepared specimens are hybridized with the probe nucleic acid that is labeled non-isotopically with, for example, a fluorescent dye, biotin or digoxigenin. The hybridized signals are then detected by fluorimetric or by enzymatic methods, for example, by using a fluorescence or light microscope. The detected signal and image can be recorded on light sensitive film.

An advantage of using a fluorescent probe is that the hybridized image can be readily analyzed using a powerful confocal microscope or an appropriate image analysis system with a charge-coupled device (CCD) camera As compared with radioactive methods, FISH offers increased sensitivity. In additional to offering positional information, FISH allows better observation of cell or tissue morphology. Because of the nonradioactive approach, FISH has become widely used for localization of specific DNA or mRNA in a specific cell or tissue type.

The in situ hybridization methods and the preparations useful herein are describe in Wu, W. et al., eds., Methods in Gene Biotechnology, CRC Press, 1997, chapter 13, pages 279-289. This book is incorporated by reference in its entirety, as are the references cited therein. A number of patents and papers that describe various in situ hybridization techniques and applications, also incorporated by reference, are: U.S. Pat. Nos. 5,912,165; 5,906,919; 5,885,531; 5,880,473; 5,871,932; 5,856,097; 5,837,443; 5,817,462; 5,784,162; 5,783,387; 5,750,340; 5,759,781; 5,707,797; 5,677,130; 5,665,540; 5,571,673; 5,565,322; 5,545,524; 5,538,869; and 5,501,954, 5,225,326, 4,888,278. Other related references include Jowett, T, Methods Cell Biol;59:63-85 (1999) Pinkel et al., Cold Spring Harbor Symp. Quant. Biol. LI:151-157 (1986); Pinkel, D. et al., Proc. Natl. Acad. Sci. (USA) 83:2934-2938 (1986); Gibson et al., Nucl. Acids Res. 15:6455-6467 (1987); Urdea et al., Nucl. Acids Res. 16:4937-4956 (1988); Cook et al., Nucl. Acids Res. 16:4077-4095 (1988); Telser et al., J. Am. Chem. Soc. 111:6966-6976 (1989); Allen et al., Biochemistry 28:4601-4607 (1989); Nederlof, P. M. et al., Cytometry 10:20-27 (1989); Nederlof, P. M. et al., Cytometry 11:126-131 (1990); Seibl, R., et al., Biol. Chem. Hoppe-Seyler 371:939-951 (October 1990); Wiegant, J. et al., Nucl. Acids Res. 19:3237-3241 (1991); McNeil J A et al., Genet Anal Tech Appl 8:41-58 (1991); Komminoth et al., Diagnostic Molecular Biology 1:85-87 (1992); Dauwerse, J G et al., Hum. Mol. Genet. 1:593-598 (1992); Ried, T. et al., Proc. Natl. Acad. Sci. (USA) 89:1388-1392 (1992); Wiegant, J. et al., Cytogenet. Cell Genet. 63:73-76 (1993); Glaser, V., Genetic. Eng. News. 16:1, 26 (1996); Speicher, M R, Nature Genet. 12:368-375 (1996).

Detection of “Unknown” Gene Product

In an extreme case, in which an upregulated DNA “X” is identified but its protein product “Y” is unknown, one would first examine the expressed DNA X sequence. The full length gene sequence may be obtained by accessing a human genomic database such as that of Celera. In either case, examination of the coding sequence for appropriate motifs will indicate whether the encoded protein Y is secreted protein or a transmembrane protein. If no antibodies specific for protein Y are already available, the peptides of protein Y can be designed and synthesized using known principles of protein chemistry and immunology. The object is to create a set of immunogenic peptides that elicit antibodies specific for epitopes of the protein that reside on its surface. Alternatively, the coding DNA or portions thereof can be expression-cloned to produce a polypeptide or peptide epitope thereof. That protein or peptide can be used as an immunogen to immunize animals for the production of antisera or to prepare monoclonal antibodies (mAbs). These polyclonal sera or mAbs can then be applied in an immunoassay, preferably an EIA, to detect the presence of protein Y or measure its concentration in a body fluid or cell/tissue sample.

Therapeutic Methods

Taking the lead from the drug discovery methods described above, one can exploit the present invention to treat CC-RCC based on the knowledge of the genes that are either up- or down-regulated in a highly predicable manner across CC-RCC cases (see Tables 2-5 in Examples). Based on the nature of the deduced protein product, one can devise a means to inhibit the action of, or remove an upregulated protein. In the case of a receptor, one would treat the upregulated receptor with an antagonist, a soluble receptor or a “decoy” ligand binding site of a receptor (Gershoni J M et al., Proc Natl Acad Sci USA, 988 85:4087-9; U.S. Pat. No. 5,770,572).

For an under-expressed receptor, an agonist or mimetic would be administered to maximize binding and activation of those receptor molecules which are expressed.

As for the set of genes that are shown here to be down-regulated in aggressive CC-RCC, one can devise a therapy targeted specifically at this form of the cancer, that would be used alone or in combination with known therapeutic approaches as discussed above. A preferred approach would be to stimulate production of the protein by administering an agent that promoted production, enhanced its stability or inhibited its degradation or metabolism. Alternatively, one could design means to bypass the metabolic step or signal pathway step that was affected by this down-regulation. This could by achieved by stimulating downstream steps in such pathways. If a receptor was involved, then, as above agonists or mimics could be used to heighten responses of cells expressing too little of the receptor.

Antibodies may be administered to a patient to bind and inactivate (or compete with) secreted protein products or expressed cell surface products of upregulated genes.

Moreover, for the down-regulated genes, gene therapy methods could be used to introduce more copies of the affected gene or more actively expressed genes operatively linked to strong promoters, e.g., inducible promoters, such as an estrogen inducible system (13raselmann, S. et al. Proc Natl Acad Sci USA (1993) 90:1657-1661). Also known are repressible systems driven by the conventional antibiotic, tetracycline (Gossen, M. et al., Proc. Natl. Acad. Sci. USA 89:5547-5551 (1992)).

In the case of upregulated genes, this approach would be extended to include antisense oligonucleotide or polynucleotide constructs that would inhibit gene expression in a highly specific manner. Multiple antisense constructs specific for different upregulated genes could be employed together. The sequences of the upregulated genes described herein are used to design the antisense oligonucleotides (Hambor, J E et al., J. Exp. Med. 168:1237-1245 (1988); Holt, J T et al., Proc. Nat'l. Acad. Sci. 83:4794-4798 (1986); Izant, J G et al., Cell 36:1007-1015 (1984); Izant, J G et al., Science 229:345-352 (1985); De Benedetti, A. et al., Proc. Natl. Acad. Sci. USA 84:658-662 (1987)). The antisense oligonucleotides may range from 6 to 50 nucleotides, and may be as large as 100 or 200 nucleotides. The oligonucleotides can be DNA or RNA or chimeric mixtures or derivatives or modified versions thereof, single-stranded or double-stranded. The oligonucleotides can be modified at the base moiety, sugar moiety, or phosphate backbone (as discussed above). The oligonucleotide may include other appending groups such as peptides, or agents facilitating transport across the cell membrane (see, e.g. Letsinger et al., 1989, Proc. Natl. Acad. Sci. USA 84:684-652; PCT Publication WO 88/09810, published December 15, 1988) or blood-brain barrier (e.g., PCT Publication No. WO 89/10134, published Apr. 25, 1988), hybridization-triggered cleavage agents (e.g. Krol et al., 1988, BioTechniques 6:958-976) or intercalating agents (e.g., Zon, 1988, Pharm. Res 5:539-549).

The therapeutic methods that require gene transfer and targeting may include virus-mediated gene transfer, for example, with retroviruses (Nabel, E. G. et al., Science 244:1342 (1989), lentiviruses, recombinant adenovirus vectors (Horowitz, M. S., In: Virology, Fields, B N et al., eds, Raven Press, New York, 1990, p. 1679, or current edition; Berkler, K L, Biotechniques 6:616 919,1988), Strauss, S E, In: The Adenoviruses, Ginsberg, H S, ed., Plenum Press, New York, 1984, or current edition), Adeno-associated virus (AAV) is also useful for human gene therapy (Samulski, R J et al., EMBO J. 10:3941 (1991); (Lebkowski, J S, et al., Mol. Cell. Biol. (1988) 8:3988-3996; Kotin, R M et al., Proc. Natl. Acad. Sci. USA (1990) 87:2211-2215); Hermonat, P L, et al., J. Virol. (1984)51:329-339). Improved efficiency is attained by the use of promoter enhancer elements in the plasmid DNA constructs (Philip, R. et al., J. Biol. Chenz. (1993) 268:16087-16090).

In addition to virus-mediated gene transfer in vivo, physical means well-known in the art can be used for direct gene transfer, including administration of plasmid DNA (Wolff et al., 1990, supra) and particle-bombardment mediated gene transfer, originally described in the transformation of plant tissue (Klein, T M et al., Nature 327:70 (1987); Christou, P. et al., Trends Biotechnol. 6:145 (1990)) but also applicable to mammalian tissues in vivo, ex vivo or in vitro (Yang, N.-S., et al., Proc. Natl. Acad. Sci. USA 87:9568 (1990); Williams, R S et al., Proc. Natl. Acad. Sci. USA 88:2726 (1991); Zelenin, A V et al., FEBS Lett. 280:94 (1991); Zelenin, A V et al., FEBS Lett. 244:65 (1989); Johnston, S. A. et al, In Vitro Cell. Dev. Biol. 27:11 (1991)). Furthermore, electroporation, a well-known means to transfer genes into cell in vitro, can be used to transfer DNA molecules according to the present invention to tissues in vivo (Titomirov, A V et al., Biochim. Biophys. Acta 1088:131 ((1991)).

Gene transfer can also be achieved using “carrier mediated gene transfer” (Wu, C H et al., J. Biol. Chem. 264:16985 (1989); Wu, G Y et al., J Biol. Chem. 263:14621 (1988); Soriano, P et al., Proc. Natl. Acad. Sci. USA 80:7128 (1983); Wang, C-Y. et al., Proc. Natl. Acad. Sci. USA 84:7851 (1982); Wilson, J. M. et al., J. Biol. Chem. 267:963 (1992)). Preferred carriers are targeted liposomes (Nicolau, C. et al., Proc: Natl. Acad. Sci. USA 80:1068 (1983); Soriano et al., supra) such as immunoliposomes, which can incorporate acylated monoclonal antibodies into the lipid bilayer (Wang et al., supra), or polycations such as asialoglycoprotein/polylysine (Wu et al., 1989, supra). Liposomes have been used to encapsulate and deliver a variety of materials to cells, including nucleic acids and viral particles (Faller, D V et al., J. Virol. (1984) 49:269-272).

Preformed liposomes that contain synthetic cationic lipids form stable complexes with polyanionic DNA (Felgner, P L, et al., Proc. Natl. Acad. Sci. USA (1987) 84:7413-7417). Cationic liposomes, liposomes comprising some cationic lipid, that contained a membrane fusion-promoting lipid dioctadecyldinethyl-ammonium-bromide (DDAB) have efficiently transferred heterologous genes into eukaryotic cells Rose, J K et al., Biotechniques (1991) 10:520-525). Cationic liposomes can mediate high level cellular expression of transgenes, or mRNA, by delivering them into a variety of cultured cell lines (Malone, R., et al., Proc. Natl. Acad. Sci. USA (1989) 86:6077-6081).

Having now generally described the invention, the same will be more readily understood through reference to the following examples which are provided by way of illustration, and are not intended to be limiting of the present invention, unless specified.

### EXAMPLE I

Patients and Tumor Samples

Tissue samples were from 29 CC-RCC patients at the University Hospital, School of Medicine, Tokushima University (Japan) who underwent radical nephrectomy. Informed consent was obtained for study of surgical specimens and clinico-pathological data. Samples were anonymized prior to the study. A part of each tumor sample was frozen in liquid nitrogen immediately following surgery and stored at −80° C.

Conventional methods were used for nucleic acid isolation and preparation. Total RNA was isolated using ISOGEN solution (Nippon Gene), and poly(A)+ RNA was isolated from total RNA using the Oligotex mRNA Mini Kit (Qiagen). Remaining tumor tissue was fixed in 10% buffered formalin, sectioned and stained with hematoxylin and eosin. The WHO International Histological Classification of Tumors was used for histological evaluation of the specimens (Sobin, L. H. et al., supra)(TNM classification described above) with standard follow up for 3.2 to 137.2 months (median 83.7 months). Clinico-pathological data are summarized in Table 1.

### EXAMPLE II

### Materials and Methods

Microarray Design

Microarrays were produced using conventional methods and materials well known in the art (Eisen et al., Methods Enzymol (1999) 303:179-205) with slight modifications. Bacterial libraries purchased from Research Genetics, Inc. were the source of 21,632 cDNAs which were PCR amplified 21,632 directly. cDNA clones were ethanol-precipitated and transferred to 384-well plates from which they were printed onto poly-l-lysine coated glass slides using a home-built robotic microarrayer (www.microarrays.org/ndfs/PrintingArrays). The boundaries of the array where then marked with a diamond scriber to discriminate the edges (diamond scriber available by catalogue, VWR #52865-005) since the array is mostly invisible after post-processing. The printed array was immersed into a humid chamber prepared with 100 ml 1×SSC and allowed to rehydrate on an inverted heat block of preferably, 70-80° C., block for about 3 seconds. The cDNA was UV crosslink to glass with Stratalinker set for about 65 mJ. (Preferably, set display to “650”, which is 650×100 μJ).

TABLE1
Patient clinical data and corresponding prognosis classifications

| | | | | | | Prognosis Group | |
|---|---|---|---|---|---|---|---|
| | | | | | Outcome | Pathology/ | Gene |
| patient | Grade | Stage | Outcome | Duration | Group | Staging | Expression |
| 46 | G1 | S1 | NED | 62.6 | L | L | L |
| 42 | G1 | S1 | NED | 77.3 | L | L | L |
| 41 | G1 | S1 | NED | 80.3 | L | L | L |
| 30 | G2 | S3 | NED | 87.1 | L | H* | H* |
| 7 | G1 | S1 | NED | 92.1 | L | L | L |
| 26 | G1 | S1 | NED | 96 | L | L | L |
| 24 | G1 | S1 | NED | 97.3 | L | L | L |
| 15 | G1 | S1 | OCD | 100.4 | L | L | L |
| 32 | G1 | S2 | OCD | 110.4 | L | L | L |
| 1 | G1 | S1 | NED | 111.6 | L | L | L |
| 21 | G1 | S1 | NED | 114.6 | L | L | L |
| 20 | G1 | S1 | NED | 115.8 | L | L | L |
| 35 | G1 | S3 | NED | 120.5 | L | H* | L |
| 9 | G1 | S3 | NED | 120.9 | L | H* | L |
| 3 | G1 | S1 | NED | 137.2 | L | L | L |
| 29 | G3 | S3 | AWC | 89.4 | L | H* | L |
| 54 | G1 | S4 | AWC | 105.6 | L | H* | L |
| 13 | G3 | S4 | Death | 3.2 | H | H | H |
| 48 | G2 | S4 | Death | 4.9 | H | H | H |
| 11 | G3 | S3 | Death | 18.8 | H | H | H |
| 60 | G3 | S4 | Death | 20.8 | H | H | H |
| 31 | G3 | S3 | Death | 22.6 | H | H | H |
| 53 | G3 | S4 | Death | 26.2 | H | H | H |
| 5 | G2 | S4 | Death | 31.7 | H | H | H |
| 12 | G2 | S4 | Death | 33.8 | H | H | H |
| 55 | G2 | S2 | Death | 55.8 | H | L* | H |
| 56 | G3 | S4 | AWC | 14.8 | U | H | L |
| 58 | G3 | S4 | AWC | 16.6 | U | H | H |
| 59 | G2 | S3 | NED | 41.1 | U | H | H |

Stage and grade information (columns 2, 3) is for primary tumor upon resection.
Outcomes (column 4) are: “no evidence of disease at last visit” (NED), “alive with cancer” (AWC), “other cause of death” (OCD) and “death” (due to cancer).
Duration (column 5) is months between nephrectomy and latest outcome assessment.
Outcome group (column 6) is the risk group based on actual patient outcome;
Pathology prognosis group (column 7) is based on staging of primary tumor;
Gene expression prognosis group (column 8) is based on molecular prognosis test based on genes in NODE 1281. Risk groups include high-risk (H), low-risk (L) and unknown (U).
*indicates deviation from actual risk group. for 20 minutes with the lid down. The array was then snap centrifugation dried (cDNA side up)

Prior to applying hybridization solution containing labeled probes (below), slides were blocked before target hybridization, using bovine serum albumin (BSA) solution (1% BSA, 5×SSC, 0.1% SDS) as described by Volpert et al., J Clin Invest (1999) 98(3): 671-679)., Blocking is preferably done within 1 hour of hybridization, most preferably immediately before.

Tissue cDNA Preparation

Samples (2 μg of poly(A)-RNA from each kidney tumor and from normal kidney tissue from the same patient were reverse transcribed with oligo (dT) primers and Superscript II (Life Technologies, Inc) in the presence of Cy5-dCTP and Cy3-dCTP (Amersham Pharmacia Biotech), respectively (Methods Enzynol (1999) 303:179-205). 10 The poly(A)-mRNA isolation procedure used by the inventors is detailed below, however, the skilled artisan will appreciate that any method of isolation and fluoro-labeling can be used. The inventors mixed 2 μg of mRNA with 2 μg of a regular or anchored oligo-dT primer in a total volume of 15 μl:

| | |
|---|---|
| Cy3 | Cy5 |

| | | | |
|---|---|---|---|
| mRNA | 2 μg | 2 μg | (Anchored: |
| | | | TTT TTT TTT TTT |
| | | | TTT TTT TTV N-3′)* |
| Oligo-dT | 2 μg | 2 μg | SEQ ID NO:498 |
| Total volume: | 15 μL | μL | |

*(“V” refers to A, G or C; “N” refers to A, G, C or T)

Next, the reaction mixture was heated to 70° C. for about 10 min and cooled on ice to which was added 15 μL of the following reaction mixture with denatured mRNA (for a total of 30 μl):

| | | Unlabeled | | |
|---|---|---|---|---|
| | | dNTPs | | |
| Reaction mixture*: | Vol μl | 100 mM | Vol μl | Final conc. |
| 5X first-strand buffer** | 6.0 | dATP | 25 | 25 mM |
| 0.1M DTT | 3.0 | dCTP | 15 | 15 mM |
| Unlabeled dNTPs | 0.6 | dGTP | 25 | 25 mM |
| Cy3 or Cy5-dCTP (1 mM, | 3.0 | dTTP | 25 | 25 mM |
| Amersham) | | | | |
| Superscript II (200 U/μL, | 2.0 | H₂O | 10 | |
| Gibco BRL) | | | | |
| H₂O | 0.4 | | | |
| Total volume: | 15 | Total | 100 | |
| | | volume: | | |

*Reaction mixture (Master Mix) available that contains buffer, DTT, dNTPs, and H₂O (combine 10 μl Master Mix with 3 μl Cy3 or Cy5 dye and 2 μl Superscript)
**5X first-strand buffer: 250 mM Tris-HCl (pH 8.3), 375 mM KCl, 15 mM MgCl2)

The combined reaction mix was incubated at 42° C. for 1.5-2hrs. RNA degradation was facilitated by the addition of 15 μl of 0.1 M NaOH, and incubation at 70° C. for 10 min. The degradation reaction was neutralized by addition of 15 μl of 0.1 M HCl, and the total volume was brought to 500 μl with TE (10 mM Tris, 1 mM EDTA).

Next, 20 μg of Cot-1 human DNA (GIBCO-BRL) was added to each sample. The target cDNAs (post RT-PCR replicons) were purified by centrifuging in a Microcon-30 micro-concentrator (Amicon, 10,000×g (rcf)for 10 min until ˜10 μl remained). Purification can be monitored by observing the concentration of the “colored probe.”

TE, 450 μl was added to each Microcon-30 unit and the retentate collected in a fresh microtube. The collected retentate from the previous step was added into the Microcon-30 unit containing the other sample in order to combine the separate probes (Cy3 and Cy5). The final volumes should be about 500 μl (if less than 500 μl, adjust with TE).

The 500 μl mix was spun with a microcon-30 (10,000×g (rcf)×12 min) containing labeled samples in order to concentrate again to a volume of less than 11 μl. Add 1 μL of 10 μg/μl polyA RNA (Sigma, #P9403) and 1 μl of 10 μg/μl tRNA (GIBCO-BRL, #15401-011) and adjust volume to 15-17 μl with distilled water.

The mixture was heated at 95° C. for 3 min. and briefly centrifuged to collect condensation. Then the denatured target was combined with equal volume of 2× hybridization solution preheated to 42° C. The mixture was heated at 95° C. for 3 min. and briefly centrifuged to collect condensation. Then the denatured target was combined with equal volume of 2× hybridization solution preheated to 42° C.

Hybridizing Microarrays Blocked with BSA

Immediately or shortly before hybridization the prepared microarray slides containing single stranded cDNA probes were BSA blocked (supra).

The 2×hybridization solution contains: 50% formamide; 10×SSC; 0.2% SDS. Final volume was 30-35 μl. The hybridization solution was incubated at 42° C. for 20-30 min. The labeled target +hybridization solution was then applied to a prepared microarray slide at 42° C. (using a hot block to preheat the slide and coverslip).

20 μl H₂O was placed in the wells of the hybridization chamber. The slide was sealed in a hybridization chamber and placed in a 42° C. water bath. Microarrays were hybridized for ˜16-20 hours.

Slides were removed from hybridization chamber and immediately placed in a first rinse station with wafer holders/forceps (5 total wash/rinse stations): Exposure of labeled probe to light is to be minimized. The rinsing protocol is detailed below:
A. 1×SSC, 0.1% SDS (376 mls dH₂O, 20 mls 20×SSC, 4 mls 10% SDS) This first rinse is carried out at 42° C. until the cover slip is washed off, keep the slide in this solution for 5 minutes. Place slide in new metal tray in the next station.
B. 0.2×SSC, 0.1% SDS (392 mls dH₂O, 4 mls 20X SSC, 4 mls 10% SDS) Gently shake station with slides and holder on rotator for 5 minutes. Take individual slides out of the tray and place in next clean station.
C. 0.2×SSC (396 mls dH₂O, 4 mls 20×SSC) Shake gently for 5 minutes. Transfer entire slide holder into next station. Carry out this step three times, using fresh solution each time. Preferably, three stations are used where repeated washing steps are carried out with fresh solution.

Slides were dried by snap centrifugation (5 min. at 550 rpm) and scanned immediately using a commercially available confocal fluorescent scanner equipped with lasers operating at 532 nm and 635 nm wavelengths. (Scan Array Lite, GSI Lumonics).

Data Analysis

Images were analyzed using the software Genepix Pro 3.0 (Axon). Spots showing no signal or obvious defects were excluded from the analysis. Hybridization signal intensities from the remaining spots had the background subtracted and were tabulated in a red-to-green ratio, representing tumor mRNA expression relative to the mRNA expression of the corresponding normal kidney tissue. Ratios were log transformed and normalized so that the average ratio equaled zero. cDNAs with non-flagged spots in 75% of the experiments and with expression ratios that varied at least 2-fold in at least 2 experiments were selected for further analysis. The ratios were median-polished as described to provide values relative to the other samples. The software programs CLUSTER and TREEVIEW were used for hierarchical clustering and visualization (http://rana.standford.edu/software).

Clusterfinder

The present inventors developed the program “CLUSTERFINDER” to identify sub-clusters of polynucleotides that best distinguish between two defined sample groups. This clustering methodology entails, averaging the polynucleotides within a subcluster so that each patient has one expression value per subcluster. These expression value averages are separated into two groups based on the user-defined criteria. Here, staging criteria and patient fatality were employed. For each group of expression value averages, means (μ) and standard deviations (σ) were calculated. The discrimination score (ds) is calculated as follows:
ds=|μ₁−μ₂|/(σ₁+σ₂)

This metric maximizes difference between the means of the two groups and minimizes the variation within groups (Golub et al., supra). The method begins with the smallest clusters (2 cDNAs) and moves through a dendrogram identifying nodes in the tree that maximize both discrimination score and cluster size.

A permuted t-test was used to assess each cDNAs individual ability to distinguish between the two groups of patients (Hedenfalk, I., et al. (2001) N Engl J Med 344:539-48). Patients were randomly assigned into two groups 10,000 times. For each random permutation, a t-statistic was generated to test expression significance for each cDNA. The distribution of t-statistics was used to define a 99.9% significance threshold (α=0.001). If the t-statistic for the real distinction exceeded the 99.9% significance threshold, the CDNA was considered predictive.

This design permitted two valuable approaches to analyze the data. First, the use of the patient-matched normal tissue as a reference, against which mRNA expression in the tumors is measured, allows identification of aberrant polynucleotide expression (up or down) in each tumor. Second, since Cy3-labeled normal tissue was a common reference in all the experiments, values obtained from different experiments could be compared directly to identify gene expression patterns that would account for clinical differences such as grade, stage or aggressiveness of the tumor.

### EXAMPLE III

### Identification of Useful Probes for Up- and Down-Regulated Genes

The inventors first sought to identify genes that were up- or down-regulated regularly in tumor tissue relative to matched normal kidney tissue. The criterion for a useful probe was one that detected a gene that is up-regulated or down-regulated at least 2-fold in at least 75% of the CC-RCC samples. The inventors identified 129 clones (up) and 168 clones (down) respectively. See Tables 2-5.

Up-regulated genes included many notable coding sequences:

| | |
|---|---|
| (1) | ceruloplasmin, |
| (2) | an EST highly similar to growth factor |
| | responsive protein, |
| (3) | nicotinamide N-methyltransferase, |
| (4) | lysyl oxidase, |
| (5) | an EST highly similar to angiopoietin-related |
| | protein, |
| (6) | tumor necrosis factor α-induced protein 6, |
| (7) | insulin-like growth factor binding protein-3 |
| (8) | enolase-2, |
| (9) | fibronectin-1 and |
| (10) | vascular endothelial growth factor (VEGF). |

Down-regulated cDNAs included:

| | |
|---|---|
| (1) | kininogen, |
| (2) | fatty acid binding protein 1, |
| (3) | phenylalanine hydroxylase, |
| (4) | epidermal growth factor, and |
| (5) | plasminogen. |

In addition, six members of the metallothionein family were down regulated and coordinately expressed across all patients.

TABLE 2
First Set of Commonly Up-Regulated Genes in CC-RCC

| | | | SEQ | | INCIDENCE | | | |
|---|---|---|---|---|---|---|---|---|
| | GENBANK | | ID | AVERAGE | % OF RCC* | | CELERA | |
| | ACCESSION # | NAME (FROM RESEARCH GENETICS DATABASE) | NO: | FOLD UP | 2-FOLD* | 3-FOLD** | NO# | E VALUE |
| 1 | H86554 | Ceruloplasmin (ferroxidase) | 40 | 16.9 | 96.2 | 96.2 | hCG21213 | 6 × 10⁻⁸⁵ |
| 2 | R00332 | ESTs, highly similar to growth factor-responsive protein, | 41 | 14.1 | 96.4 | 96.4 | hCG41109 | 2 × 10⁻⁶² |
| | | vascular smooth muscle [R. norvegicus] | | | | | | |
| 3 | T72235 | Nicotinamide N-methyltransferase | 42 | 13.5 | 96.6 | 96.6 | hCG39357 | 10⁻¹⁰² |
| 4 | W72051 | Fatty acid binding protein 7, brain | 43 | 13.2 | 87.5 | 75.0 | hCG19286 | 10⁻¹⁸⁰ |
| 5 | W70343 | Lysyl oxidase | 44 | 11.2 | 95.8 | 87.5 | hCG37363 | 10⁻⁷⁵ |
| 6 | H99075 | ESTs | 44 | 10.7 | 95.7 | 87.0 | hCG37363 | 0 |
| 7 | W30988 | ESTs, highly similar to angiopoietin-related | 45 | 11.1 | 100.0 | 100.0 | hCG23958 | 10⁻¹¹¹ |
| 8 | T54298 | protein [H. sapiens] | 45 | 8.1 | 100.0 | 96.6 | hCG23958 | 10⁻¹⁵³ |
| 9 | N50654 | Ceruloplasmin (ferroxidase) | 46 | 10.6 | 95.8 | 95.8 | hCG21214 | 10⁻¹⁴⁶ |
| 10 | W93163 | Tumor necrosis factor, α-induced protein 6 | 47 | 10.5 | 100.0 | 100.0 | hCG41965 | 6 × 10⁻⁷⁶ |
| 11 | AA598601 | Insulin-like growth factor binding protein 3 | 48 | 7.6 | 96.6 | 89.7 | hCG18013 | 0 |
| 12 | AA678335 | H. sapiens phosphodiesterase I/nucleotide | 49 | 7.6 | 84.0 | 84.0 | hCG18059 | 10⁻¹⁴⁵ |
| | | yrophosphatase 3 (PDNP3) mRNA | | | | | | |
| 13 | AA164819 | ESTs | 50 | 7.1 | 96.3 | 88.9 | hCG38036 | 0 |
| 14 | AA485896 | | 50 | 6.8 | 96.4 | 89.3 | hCG38036 | 10⁻¹⁴⁵ |
| 15 | N26171 | ESTs | 51 | 6.2 | 87.5 | 79.2 | hCG19701 | 0 |
| 16 | AA487787 | Von Willebrand factor | 52 | 6.2 | 100.0 | 87.5 | hCG24322 | 10⁻¹⁶⁸ |
| 17 | AA450189 | Enolase 2, (γ, neuronal) | 53 | 6.0 | 96.4 | 92.9 | hCG25937 | 0 |
| 18 | R62612 | Fibronectin 1 | 54 | 5.6 | 93.1 | 79.3 | hCG16692 | 2 × 10⁻⁵¹ |
| 19 | H20872 | FcγIIIaR (CD16); low affinity receptor | 55 | 5.5 | 85.7 | 82.1 | hCG16608 | 0 |
| | | for IgG Fc fragment | | | | | | |
| 20 | W72293 | ESTs | 56 | 5.5 | 93.1 | 89.7 | hCG20029 | 2 × 10⁻⁵⁸ |
| 21 | AA055835 | Caveolin 1, caveolae protein, 22 kD | 57 | 5.4 | 92.9 | 75.0 | hCG39088 | 10⁻¹²¹ |
| 22 | AA873159 | Apolipoprotein C-I | 58 | 5.3 | 88.9 | 81.5 | hCG22139 | 4 × 10⁻⁶⁸ |
| 23 | AA017544 | Regulator of G-protein signalling 1 | 59 | 5.2 | 85.7 | 82.1 | hCG39901 | 10⁻¹⁷⁹ |
| 24 | R19956 | Vascular endothelial growth factor | 60 | 5.1 | 96.4 | 85.7 | hCG18998 | 4 × 10⁻⁹⁰ |
| 25 | H99816 | Procollagen-lysine, 2-oxoglutarate 5-dioxygenase | 61 | 5.1 | 96.4 | 78.6 | hCG16089 | 10⁻¹⁴⁹ |
| | | (lysine hydroxylase) 2 | | | | | | |
| 26 | R49597 | ESTs | 62 | 4.6 | 95.8 | 75.0 | hCG15938 | 4 × 10⁻⁸³ |
| 27 | AA405000 | H. sapiens ribonuclease 6 precursor mRNA | 63 | 4.5 | 96.2 | 80.8 | hCG15018 | 10⁻¹⁴⁹ |
| 28 | H58873 | Solute carrier family 2 (facilitated glucose transporter), | 64 | 4.5 | 93.1 | 86.2 | hCG23157 | 10⁻¹⁷⁶ |
| | | member 1 | | | | | | |
| 29 | T62491 | Chemokine (C-X-C motif), receptor 4 (fusin) | 65 | 4.4 | 89.7 | 75.9 | hCG25754 | 10⁻¹²⁶ |
| 30 | AA443899 | DC36 (collagen type I receptor, thrombospondin | 66 | 4.2 | 89.3 | 75.0 | hCG25301 | 0 |
| | | receptor-like 1 | | | | | | |
| 31 | AI004331 | Human MHC class II HLA-DQβ mRNA (DR7 DQw2), | 67 | 4.1 | 85.7 | 78.6 | CG201516 | 0 |
| | | complete cds | | | | | | |
| 32 | AA488892 | ESTs, Weakly similar to gag-pol polyprotein | 68 | 4.0 | 85.7 | 75.0 | hCG95780 | 5 × 10⁻⁸⁹ |
| | | [M. musculus] | | | | | | |

*The values in this column are the % of CC-RCC patients in whom a given gene was expressed at least 3-fold higher compared to control kidney tissue. Genes included in this Table met or exceeded this threshold in at least 75% of CC-RCC patients.
**The values in this column are the % of CC-RCC patients in whom a given gene was expressed at least 2-fold higher compared to control kidney tissue. Genes included in this Table met or exceeded this threshold in at least 75% of CC-RCC patients.

TABLE 3
Second Set of Commonly Up-Regulated Genes in CC-RCC

| | | | SEQ | AVERAGE | |
|---|---|---|---|---|---|
| | GENBANK | | ID | FOLD | INCIDENCE |
| | ACCESSION # | NAME (FROM RESEARCH GENETICS DATABASE) | NO: | UP | % OF RCC* |
| 1 | AA101875 | Chondroitin sulfate proteoglycan 2 (versican) | 140 | 5.5 | 80.8 |
| 2 | W60845 | Cell division cycle 42 (GTP-binding protein, 25 kD) | 141 | 4.8 | 77.8 |
| 3 | AA457700 | Cytochrome b-561 | 142 | 4.8 | 88.9 |
| 4 | H95819 | ESTs | 143 | 4.7 | 91.3 |
| 5 | AA136707 | Procollagen-lysine, 2-oxoglutarate | 144 | 4.5 | 96.4 |
| | | 5-dioxygenase (lysine hydroxylase) 2 | | | |
| 6 | R43605 | KIAA0293 protein | 145 | 4.4 | 93.1 |
| 7 | N63943 | Lysozyme (renal amyloidosis) | 146 | 4.2 | 82.8 |
| 8 | N76878 | Decidual protein induced by progesterone | 147 | 4.1 | 86.2 |
| 9 | R95749 | ESTs | 148 | 4.1 | 88.9 |
| 10 | AA417622 | ESTs | 149 | 4.0 | 92.9 |
| 11 | AA460224 | ESTs | 150 | 3.9 | 92.6 |
| 12 | AA460152 | Serum-inducible kinase | 151 | 3.9 | 86.2 |
| 13 | W72329 | Lymphotoxin α (TNF superfamily, member 1) | 152 | 3.9 | 82.1 |
| 14 | AA700054 | Adipose differentiation-related protein; adipophilin | 153 | 3.9 | 86.2 |
| 15 | W80701 | ESTs, Weakly similar to HERV-E envelope | 154 | 3.8 | 91.7 |
| | | glycoprotein [H. sapiens] | | | |
| 16 | AA442984 | major histocompatibility complex (MHC) class II, DQβ1 | 155 | 3.8 | 86.2 |
| 17 | H12338 | TYRO protein tyrosine kinase binding protein | 156 | 3.8 | 89.7 |
| 18 | AA629189 | keratin 4 | 157 | 3.8 | 85.7 |
| 19 | AA176581 | myoglobin | 158 | 3.8 | 82.1 |
| 20 | R33363 | decidual protein induced by progesterone | 159 | 3.7 | 93.1 |
| 21 | AA456821 | ESTs, Weakly similar to intrinsic factor-B12 | 160 | 3.7 | 87.0 |
| | | receptor precursor [H. sapiens] | | | |
| 22 | R43734 | laminin, α4 | 161 | 3.7 | 82.1 |
| 23 | N38801 | ESTs, Highly similar to Complement C1q | 162 | 3.7 | 89.7 |
| | | subcomponent, C chain precursor | | | |
| 24 | AA458472 | MHC, class II, DQ β 1 | 163 | 3.6 | 79.3 |
| 25 | AA489611 | lactate dehydrogenase A | 164 | 3.5 | 89.7 |
| 26 | N90491 | ESTs, Highly similar to Complement C1q | 165 | 3.5 | 88.9 |
| | | subcomponent, C chain precursor | | | |
| 27 | N30205 | ESTs | 166 | 3.4 | 79.3 |
| 28 | R47979 | Human HLA-DR α-chain mRNA | 167 | 3.4 | 75.9 |
| 29 | T62849 | ESTs | 168 | 3.4 | 81.8 |
| 30 | AA425450 | glycoprotein (transmembrane) nmb | 169 | 3.4 | 81.5 |
| 31 | N94616 | laminin, α 4 | 170 | 3.3 | 77.8 |
| 32 | AA478542 | A kinase (PRKA) anchor protein (gravin) 12 | 171 | 3.3 | 79.3 |
| 33 | AA236164 | cathepsin S | 172 | 3.3 | 85.7 |
| 34 | AA677340 | phosphorylase kinase, α 2 (liver) | 173 | 3.3 | 82.1 |
| 35 | AA002126 | apoptosis inhibitor 2 | 174 | 3.3 | 84.0 |
| 36 | AA486627 | MHC class II, DPβ1 | 175 | 3.3 | 75.9 |
| 37 | AA486567 | ESTs | 176 | 3.3 | 91.7 |
| 38 | W37864 | phosphatase and tensin homolog | 177 | 3.3 | 88.9 |
| | | (mutated in multiple advanced cancers 1) | | | |
| 39 | H15662 | KIAA0291 protein | 178 | 3.3 | 80.8 |
| 40 | N71028 | ESTs | 179 | 3.3 | 82.8 |
| 41 | AA421296 | CD68 antigen | 180 | 3.3 | 82.8 |
| 42 | W60701 | MHC class I, A | 181 | 3.2 | 75.9 |
| 43 | AA599138 | ESTs | 182 | 3.2 | 89.3 |
| 44 | AA634028 | Human mRNA for SB class II MHC α-chain | 183 | 3.2 | 79.3 |
| 45 | AA682558 | ESTs | 184 | 3.2 | 81.8 |
| 46 | AA132090 | CD53 antigen | 185 | 3.2 | 81.5 |
| 47 | R97251 | Homo sapiens clone 24655 mRNA sequence | 186 | 3.1 | 85.7 |
| 48 | H79353 | Fc fragment of IgE, high affinity I, receptor for; γ polypeptide | 187 | 3.1 | 85.7 |
| 49 | W73144 | lymphocyte cytosolic protein 1 (L-plastin) | 188 | 3.1 | 79.3 |
| 50 | AI005515 | hexokinase 2 | 189 | 3.1 | 81.8 |
| 51 | AA478585 | butyrophilin, subfamily 3, member A3 | 190 | 3.0 | 75.9 |
| 52 | AA126982 | sin3-associated polypeptide, 30 kD | 191 | 3.0 | 80.0 |
| 53 | AA644657 | MHC class I, A | 192 | 3.0 | 82.8 |
| 54 | AA425806 | Suppressin (nuclear deformed epidermal | 193 | 3.0 | 78.6 |
| | | autoregulatory factor-1 (DEAF-1)-related) | | | |
| 55 | T89391 | Caveolin 2 | 194 | 3.0 | 78.6 |
| 56 | H11732 | ESTs | 195 | 3.0 | 79.2 |
| 57 | AA083407 | Stimulated trans-acting factor (50 kDa) | 196 | 3.0 | 77.8 |
| 58 | AA702254 | MHC, class II, DN α | 197 | 2.9 | 82.8 |
| 59 | W88967 | MHC, class II, DR β 1 | 198 | 2.9 | 75.9 |
| 60 | AA491191 | interferon, γ-inducible protein 16 | 199 | 2.9 | 82.1 |
| 61 | AA157813 | interferon, α-inducible protein 27 | 200 | 2.9 | 76.9 |
| 62 | AA777488 | ESTs, moderately similar to | 201 | 2.9 | 75.9 |
| | | glyceraldehyde-3-phosphate dehydrogenase, liver | | | |
| 63 | N66053 | Butyrophilin, subfamily 3, member A1 | 202 | 2.9 | 75.9 |
| 64 | AA988615 | Human HLA-F gene | 203 | 2.8 | 78.6 |
| 65 | N32226 | ESTs | 204 | 2.8 | 77.8 |
| 66 | T63324 | MHC, class II, DQ α1 | 205 | 2.8 | 75.9 |
| 67 | AA669055 | MHC, class II, DQ β1 | 206 | 2.8 | 79.3 |
| 68 | AA102068 | heat shock transcription factor 4 | 207 | 2.8 | 78.6 |
| 69 | H95960 | secreted protein, acidic, cysteine-rich (osteonectin) | 208 | 2.8 | 75.9 |
| 70 | T84762 | Homo sapiens mRNA; cDNA DKFZp434O071 | 209 | 2.8 | 85.2 |
| | | (from clone DKFZp434O071) | | | |
| 71 | AA664195 | MHC, class II, DR β1 | 210 | 2.8 | 75.0 |
| 72 | AA453978 | GM2 ganglioside activator protein | 211 | 2.7 | 84.0 |
| 73 | AA464246 | MHC, class I, C | 212 | 2.7 | 82.8 |
| 74 | H26176 | Homo sapiens mRNA; cDNA DKFZp564E1616 | 213 | 2.7 | 77.8 |
| | | (from clone DKFZp564E1616) | | | |
| 75 | AA456063 | Homo sapiens mRNA for hCRNN4, complete cds | 214 | 2.7 | 78.6 |
| 76 | AA708621 | ESTs, [H. sapiens] | 215 | 2.7 | 89.3 |
| 77 | AA284954 | colony stimulating factor 1 receptor, formerly | 216 | 2.7 | 85.2 |
| | | McDonough feline sarcoma viral (v-fms) | | | |
| | | oncogene homolog | | | |
| 78 | AA521292 | ATP-binding cassette, sub-family A (ABC1), member 1 | 217 | 2.7 | 80.8 |
| 79 | R92609 | colony stimulating factor 1 receptor, formerly | 218 | 2.6 | 77.3 |
| | | McDonough feline sarcoma viral (v-fms) | | | |
| | | oncogene homolog | | | |
| 80 | R33609 | ESTs | 219 | 2.6 | 82.8 |
| 81 | AA430540 | collagen, type IV, α 2 | 220 | 2.6 | 79.3 |
| 82 | H41165 | Ribosomal protein S19 | 221 | 2.6 | 82.8 |
| 83 | AA485371 | Bone marrow stromal cell antigen 2 | 222 | 2.5 | 75.9 |
| 84 | AA461309 | ESTs, Weakly similar to predicted | 223 | 2.5 | 75.0 |
| | | using Genefinder [C. elegans] | | | |
| 85 | AA130874 | ESTs, Weakly similar to [H. sapiens] | 224 | 2.5 | 75.9 |
| 86 | T62048 | Complement component 1, s subcomponent | 225 | 2.5 | 76.0 |
| 87 | T69304 | TAP binding protein (tapasin) | 226 | 2.5 | 79.2 |
| 88 | AA463188 | Putative serine-threonine protein kinase | 227 | 2.5 | 81.5 |
| 89 | W37721 | ESTs | 228 | 2.4 | 75.9 |
| 90 | AA862434 | Proteasome (macropain) subunit, β type, 9 | 229 | 2.4 | 75.0 |
| | | (large multifunctional protease 2) | | | |
| 91 | R22412 | Platelet/endothelial cell adhesion | 230 | 2.4 | 75.9 |
| | | molecule (CD31 antigen) | | | |

*The values in this column are the % of CC-RCC patients in whom a given gene was expressed at least 2-fold higher compared to control kidney tissue. Genes included in this Table met or exceeded this threshold in at least 75% of CC-RCC patients (but did not exceed the threshold of 3 fold upregulation in this percentage of patients

TABLE 4
First Set of Commonly Down-Regulated Genes in CC-RCC

| | GENBANK | | SEQ | AVERAGE | INCIDENCE | | | |
|---|---|---|---|---|---|---|---|---|
| | ACCESSION | NAME | ID | FOLD | % OF RCC* | | CELERA | |
| | # | (FROM RESEARCH GENETICS DATABASE) | NO: | DOWN | 2-FOLD* | 3-FOLD** | NO# | E VALUE^{§} |
| 1 | R89067 | Kininogen | 69 | 27.2 | 100.0 | 100.0 | hCG16151 | 10⁻¹⁶¹ |
| 2 | AA705692 | ESTs | 69 | 18.0 | 100.0 | 100.0 | hCG16151 | 0 |
| 3 | T53220 | Fatty acid binding protein 1, liver | 70 | 22.8 | 95.8 | 95.8 | hCG32947 | 10⁻⁴⁹ |
| 4 | AA682293 | Phenylalanine hydroxylase | 71 | 20.4 | 96.0 | 96.0 | hCG21871 | 0 |
| 5 | AA954947 | Epidermal growth factor (β-urogastrone) | 72 | 15.0 | 100.0 | 100.0 | hCG19911 | 0 |
| 6 | H72098 | aldolase B, fructose-bisphosphate | 73 | 13.6 | 100.0 | 96.6 | hCG27655 | 0 |
| 7 | AA411988 | EST | 74 | 13.3 | 100.0 | 96.4 | hCG28257 | 0.014 |
| 8 | T73187 | Plasminogen | 75 | 12.0 | 100.0 | 89.3 | hCG32944 | 3 × 10⁻⁵⁶ |
| 9 | T51617 | solute carrier family 22 (organic cation | 75 | 11.8 | 96.4 | 92.9 | hCG32944 | 10⁻¹⁶⁹ |
| | | transporter, member 3) | | | | | | |
| 10 | AA777384 | ESTs | 76 | 11.0 | 96.2 | 92.3 | hCG17572 | 0 |
| 11 | H53340 | Metallothionein 1G | 77 | 10.0 | 100.0 | 93.1 | hCG40931 | 7 × 10⁻⁶⁴ |
| 12 | AA844930 | Glycoprotein 2 (zymogen granule membrane) | 78 | 9.6 | 100.0 | 96.6 | hCG34445 | 2 × 10⁻⁸⁹ |
| 13 | | | 79 | 9.4 | 96.6 | 96.6 | | |
| 14 | AA858026 | protein C inhibitor-plasminogen activator inhibitor 3 | 80 | 9.4 | 100.0 | 96.6 | hCG40087 | 4 × 10⁻⁴ |
| 15 | H18950 | ESTs, similar to hepatocyte nuclear factor 4 γ | 81 | 9.2 | 100.0 | 96.6 | hCG40025 | 10⁻¹⁶⁹ |
| | | [H. sapiens] | | | | | | |
| 16 | | | 82 | 8.9 | 96.6 | 96.6 | | |
| 17 | AA040387 | X-prolyl aminopeptidase (aminopeptidase P) 2, | 83 | 8.8 | 96.4 | 96.4 | hCG20708 | 10⁻¹³³ |
| | | membrane-bound | | | | | | |
| 18 | H77766 | Metallothionein 1H | 84 | 8.4 | 96.6 | 86.2 | hCG23909 | 10⁻¹⁵¹ |
| 19 | N55459 | RNA helicase-related protein | 84 | 6.9 | 96.6 | 79.3 | hCG23909 | 6 × 10⁻⁹⁷ |
| 20 | H72722 | ESTs, similar to metallothionein-IB [H. sapiens] | 84 | 5.2 | 86.2 | 75.9 | hCG23909 | 10⁻⁶¹ |
| 21 | W16424 | ESTs | 85 | 8.4 | 92.6 | 85.2 | hCG41126 | 0 |
| 22 | H88329 | Calbindin 1, (28 kD) | 86 | 8.1 | 100.0 | 92.9 | hCG33059 | 10⁻¹⁴⁶ |
| 23 | N62179 | Methylmalonate-semialdehyde dehydrogenase | 87 | 7.9 | 100.0 | 96.4 | hCG21723 | 10⁻¹⁷⁹ |
| 24 | AA460298 | | 87 | 4.4 | 91.7 | 75.0 | hCG21723 | 0 |
| 25 | AA775872 | Glypican 3 | 88 | 7.9 | 100.0 | 100.0 | hCG14619 | 10⁻¹³³ |
| 26 | AA457718 | H. sapiens mRNA; cDNA DKFZp564B076 (from | 89 | 7.8 | 95.7 | 87.0 | hCG18130 | 0 |
| | | clone DKFZp564B076) | | | | | | |
| 27 | R24266 | Growth factor receptor-bound protein 14 | 90 | 7.1 | 80.8 | 76.9 | hCG40120 | 10⁻¹⁰⁴ |
| 28 | R54778 | Collagen, type XVI, α1 | 91 | 7.1 | 100.0 | 95.8 | hCG41613 | 10⁻¹⁰¹ |
| 29 | AA702640 | DOPA decarboxylase (aromatic | 92 | 7.0 | 96.3 | 85.2 | hCG18339 | 0 |
| | | L-amino acid decarboxylase) | | | | | | |
| 30 | AA664180 | Glutathione peroxidase 3 (plasma) | 93 | 6.6 | 92.9 | 85.7 | hCG39155 | 2 × 10⁻⁷⁴ |
| 31 | R10382 | Protein C inhibitor (plasminogen | 94 | 6.4 | 92.6 | 88.9 | hCG16021 | 0.058 |
| | | activator inhibitor 3 (PAI-3) | | | | | | |
| 32 | AA227594 | Mal, T-cell differentiation protein | 95 | 6.3 | 100.0 | 86.2 | hCG38742 | 10⁻¹⁴¹ |
| 33 | H68509 | UDP glycosyltransferase-2 family, polypeptide B10 | 96 | 6.1 | 95.5 | 86.4 | hCG41481 | 0.25 |
| 34 | AA676466 | Argininosuccinate synthetase | 97 | 6.1 | 96.4 | 85.7 | hCG40893 | 0 |
| 35 | H96140 | acyl-coenzyme A dehydrogenase, short/branched | 98 | 6.0 | 96.0 | 96.0 | hCG40572 | 10⁻¹⁸ |
| | | chain | | | | | | |
| 36 | H11346 | Aldehyde dehydrogenase 4 (glutamate | 99 | 6.0 | 92.9 | 89.3 | hCG25108 | 0 |
| | | γ-semialdehyde dehydrogenase; | | | | | | |
| | | pyrroline-5-carboxylate | | | | | | |
| | | dehydrogenase) | | | | | | |
| 37 | AA862999 | calcium-sensing receptor (hypercalcemia 1, | 100 | 6.0 | 100.0 | 92.9 | hCG14928 | 0 |
| | | severe neonatal hyperparathyroidism) | | | | | | |
| 38 | AA497001 | ESTs, weakly similar to BcDNA.GH02901 | 101 | 6.0 | 96.3 | 88.9 | hCG29639 | 0 |
| | | [D. melanogaster] | | | | | | |
| 39 | AA449780 | EST | 102 | 5.9 | 88.9 | 77.8 | hCG32613 | 2 × 10⁻³⁶ |
| 40 | H11369 | aldehyde dehydrogenase 4 (glutamate | 103 | 5.8 | 92.9 | 85.7 | hCG37443 | 10⁻¹⁵¹ |
| | | γ-semialdehyde dehydrogenase; | | | | | | |
| | | pyrroline-5-carboxylate | | | | | | |
| | | dehydrogenase) | | | | | | |
| 41 | AA704995 | Putative glycine-N-acyltransferase | 104 | 5.6 | 92.9 | 78.6 | hCG38673 | 0 |
| 42 | T94781 | Potassium inwardly-rectifying channel, | 105 | 5.6 | 92.9 | 78.6 | hCG22477 | 0.07 |
| | | subfamily J, member 15 | | | | | | |
| 43 | N89673 | ESTs | 106 | 5.6 | 92.6 | 81.5 | hCG39647 | 5 × 10⁻⁵⁹ |
| 44 | H37880 | ESTs, | 107 | 5.6 | 96.3 | 88.9 | hCG29091 | 0 |
| 45 | AA663884 | synaptosomal-associated protein, 25 kD | 108 | 5.5 | 95.7 | 87.0 | hCG40236 | 3 × 10⁻⁸⁸ |
| 46 | R25818 | aldehyde dehydrogenase 9 (γ-aminobutyraldehyde | 109 | 5.5 | 100.0 | 91.3 | hCG21745 | 10⁻¹⁴² |
| | | dehydrogenase, E3 isozyme) | | | | | | |
| 47 | AA700604 | Sorbitol dehydrogenase | 110 | 5.4 | 92.6 | 85.2 | hCG96145 | 0.36 |
| 48 | W95082 | Hydroxysteroid (11-β) dehydrogenase 2 | 111 | 5.4 | 96.6 | 89.7 | hCG27201 | 10⁻¹³² |
| 49 | AA677655 | Klotho | 112 | 5.4 | 92.3 | 80.8 | hCG32197 | 0 |
| 50 | N80129 | metallothionein 1L | 113 | 5.3 | 86.2 | 79.3 | hCG24714 | 10⁻¹¹¹ |
| 51 | AA402915 | aminoacylase 1 | 114 | 5.3 | 96.3 | 85.2 | hCG42576 | 4 × 10⁻⁹⁸ |
| 52 | AA863424 | dipeptidase 1 (renal) | 115 | 5.2 | 93.1 | 82.8 | hCG18560 | 0.05 |
| 53 | N78083 | Glycine dehydrogenase (decarboxylating; glycine | 116 | 5.1 | 96.4 | 92.9 | hCG31017 | 9 × 10⁻⁶⁹ |
| | | decarboxylase, glycine cleavage system protein P) | | | | | | |
| 54 | R06601 | ESTs, Moderately similar to metallothionein-II | 117 | 5.1 | 82.8 | 75.9 | hCG39693 | 4 × 10⁻³⁴ |
| | | [H. sapiens] | | | | | | |
| 55 | AA872383 | Metallothionein 1E (functional) | 117 | 4.8 | 82.8 | 75.9 | hCG39693 | 10⁻⁸⁶ |
| 56 | AA131240 | ESTs | 118 | 5.0 | 92.0 | 80.0 | hCG14827 | 0.02 |
| 57 | AA485965 | Succinate-CoA ligase, GDP-forming, α subunit | 119 | 4.9 | 92.9 | 89.3 | hCG33938 | 10⁻¹¹⁴ |
| 58 | AA196287 | ESTs, weakly similar to alternatively spliced | 120 | 4.9 | 96.6 | 89.7 | hCG21724 | 0 |
| | | product using exon 13A [H. sapiens] | | | | | | |
| 59 | R61229 | glycine amidinotransferase (L-arginine:glycine | 121 | 4.8 | 82.8 | 75.9 | hCG38743 | 10⁻¹³⁸ |
| | | amidinotransferase) | | | | | | |
| 60 | N23898 | G protein-coupled receptor kinase 2 | 122 | 4.8 | 92.9 | 82.1 | hCG20632 | 4 × 10⁻⁸⁹ |
| | | (Drosophila-like) | | | | | | |
| 61 | AA699427 | Fructose-bisphosphatase 1 | 123 | 4.7 | 93.1 | 82.8 | hCG32887 | 0 |
| 62 | | | 124 | 4.7 | 96.2 | 84.6 | | |
| 63 | AA873355 | ATPase, Na+/K+ transporting, α 1 polypeptide | 125 | 4.7 | 100.0 | 93.1 | hCG37943 | 10⁻¹⁰⁵ |
| 64 | AI000188 | UDP glycosyltransferase 2 family, polypeptide B7 | 126 | 4.6 | 85.7 | 75.0 | hCG40932 | 2 × 10⁻⁵¹ |
| 65 | N53031 | UDP glycosyltransferase 2 family, polypeptide B4 | 126 | 4.0 | 86.2 | 75.9 | hCG40932 | 2 × 10⁻¹² |
| 66 | AA459197 | Sodium channel, nonvoltage-gated 1 α | 127 | 4.6 | 89.7 | 75.9 | hCG24314 | 0 |
| 67 | W86431 | Protein C inhibitor (PAI-3) | 128 | 4.4 | 100.0 | 76.9 | hCG22335 | 8 × 10⁻⁵ |
| 68 | T65482 | L-3-hydroxyacyl-Coenzyme A dehydrogenase, | 129 | 4.4 | 96.2 | 76.9 | hCG19900 | 10⁻¹²⁷ |
| | | short chain | | | | | | |
| 69 | AA457374 | DKFZP586B0319 protein | 130 | 4.3 | 91.7 | 75.0 | hCG20212 | 0 |
| 70 | R33037 | ESTs | 131 | 4.3 | 92.0 | 76.0 | hCG18871 | 10⁻¹⁴⁰ |
| 71 | AA437099 | ESTs | 132 | 4.3 | 85.2 | 77.8 | hCG18095 | 0 |
| 72 | W01011 | SA (rat hypertension-associated) homolog | 133 | 4.2 | 89.7 | 75.9 | hCG37242 | 4 × 10⁻⁸⁴ |
| 73 | R16596 | EST, moderately similar to Cd-7 | 134 | 4.1 | 86.2 | 75.9 | hCG21792 | 2 × 10⁻⁶⁷ |
| | | Metallothionein-2 [H. sapiens] | | | | | | |
| 74 | AA863449 | Oviductal glycoprotein 1, 120 kD | 135 | 4.0 | 92.9 | 75.0 | HCG39984 | 10⁻¹⁴¹ |
| 75 | AA458884 | S100 calcium-binding protein A2 | 136 | 4.0 | 92.9 | 75.0 | hCG15472 | 6 × 10⁻⁹⁰ |
| 76 | AA608575 | Propionyl coenzyme A carboxylase, α polypeptide | 137 | 3.8 | 89.7 | 75.9 | hCG24579 | 10⁻¹¹⁶ |
| 77 | H18608 | Solute carrier family 22 (organic anion | 138 | 3.6 | 89.3 | 78.6 | hCG21316 | 10⁻¹⁴¹ |
| | | transporter), member 8 | | | | | | |

*The values in this column are the % of CC-RCC patients in whom a given gene was expressed at least 3-fold lower compared to control kidney tissue. Genes included in this Table met or exceeded this threshold in at least 75% of CC-RCC patients.
**The values in this column are the % of CC-RCC patients in whom a given gene was expressed at least 2-fold lower compared to control kidney tissue. Genes included in this Table met or exceeded this threshold in at least 75% of CC-RCC patients.
^{§}The E Value is a statistical value reflecting the probability that the match between the probe sequence and the sequence in the Celera database is due to chance alone. Thus very low values indicate virtual certainty that the sequence being queried corresponds to the particular gene in the database.

TABLE 5
Second Set Commonly Down-Regulated Genes in CC-RCC

| | | | SEQ | AVERAGE | INCIDENCE |
|---|---|---|---|---|---|
| | GENBANK | | ID | FOLD | % OF |
| | ACCESSION # | NAME (FROM RESEARCH GENETICS DATABASE) | NO: | Down | RCC* |
| 1 | AA454810 | Membrane component, chrom. 1, surface marker 1 | 231 | 5.2 | 88.5 |
| | | (40 kD glycoprotein, identified by monoclonal | | | |
| | | antibody GA733) | | | |
| 2 | N73241 | Solute carrier family 17 (sodium phosphate), member 1 | 232 | 5.2 | 91.7 |
| 3 | W85851 | ESTs | 233 | 5.2 | 75.9 |
| 4 | AA047666 | Flavin containing monooxygenase 1 | 234 | 4.9 | 82.1 |
| 5 | AA455632 | Human chromosome 3p21.1 gene sequence, complete cds | 235 | 4.8 | 89.3 |
| 6 | R98851 | Membrane metallo-endopeptidase (neutral | 236 | 4.6 | 82.8 |
| | | endopeptidase, enkephalinase, CALLA, CD10) | | | |
| 7 | N74679 | Homo sapiens mRNA for G3a protein (located | 237 | 4.6 | 85.2 |
| | | in the class III region of the MHC | | | |
| 8 | N51498 | ESTs | 238 | 4.6 | 82.6 |
| 9 | R60170 | ESTs | 239 | 4.5 | 75.0 |
| 10 | AA460012 | Solute carrier family 22 (organic cation | 240 | 4.4 | 95.7 |
| | | transporter), member 3 | | | |
| 11 | R42433 | H. sapiens mRNA for protein tyrosine phosphatase | 241 | 4.4 | 87.0 |
| 12 | R83190 | ESTs, similar to alanine-glyoxylate aminotransferase | 242 | 4.3 | 88.5 |
| | | 2 precursor from rat (R. norvegicus) | | | |
| 13 | R97050 | ESTs | 243 | 4.2 | 85.7 |
| 14 | T67549 | Plasminogen | 244 | 4.2 | 86.4 |
| 15 | AA862465 | Alpha-2-glycoprotein 1, zinc | 245 | 4.1 | 93.1 |
| 16 | H08720 | ESTs | 246 | 4.1 | 96.4 |
| 17 | N35592 | ESTs | 247 | 4.1 | 93.1 |
| 18 | AA284067 | ESTs | 248 | 4.0 | 92.6 |
| 19 | AA447115 | Stromal cell-derived factor 1 | 249 | 4.0 | 79.2 |
| 20 | T50788 | UDP glycosyltransferase 2 family, polypeptide B15 | 250 | 4.0 | 85.2 |
| 21 | AA448710 | ESTs | 251 | 4.0 | 77.8 |
| 22 | AA480851 | Claudin 10 | 252 | 3.8 | 89.3 |
| 23 | AA099593 | KIAA0977 protein | 253 | 3.8 | 88.9 |
| 24 | T58958 | Betaine-homocysteine methyltransferase | 254 | 3.8 | 79.3 |
| 25 | AA872602 | Parathyroid hormone receptor 1 | 255 | 3.8 | 93.1 |
| 26 | R44346 | ESTs, Weakly similar to T27A1.5 [C. elegans] | 256 | 3.8 | 86.2 |
| 27 | AA452278 | Solute carrier family 4, sodium bicarbonate cotransporter, member 4 | 257 | 3.7 | 85.2 |
| 28 | N74025 | Homo sapiens deiodinase, iodothyronine, type I (DIO1) mRNA | 258 | 3.7 | 92.3 |
| 29 | AA757672 | ESTs | 259 | 3.6 | 100.0 |
| 30 | AA135958 | ESTs | 260 | 3.6 | 75.9 |
| 31 | AA455800 | Gamma-glutamyl hydrolase (conjugase, folylpoly-γ-glutamyl hydrolase) | 261 | 3.6 | 82.1 |
| 32 | AA504160 | ATPase, H+ transporting, lysosomal (vacuolar proton pump), | 262 | 3.6 | 95.5 |
| | | α polypeptide, 70 kD, isoform 1 | | | |
| 33 | AA044205 | ESTs | 263 | 3.6 | 80.0 |
| 34 | H90507 | Plasminogen | 264 | 3.5 | 82.1 |
| 35 | AA427619 | ESTs, Weakly similar to α 1,2-mannosidase IB [H. sapiens] | 265 | 3.5 | 91.7 |
| 36 | W02265 | Translational inhibitor protein p14.5 | 266 | 3.5 | 75.9 |
| 37 | H17921 | ESTs | 267 | 3.5 | 92.0 |
| 38 | R76505 | ESTs | 268 | 3.5 | 96.4 |
| 39 | AA476258 | ESTs | 269 | 3.5 | 87.5 |
| 40 | N91990 | phytanoyl-CoA hydroxylase (Refsum disease) | 270 | 3.5 | 88.0 |
| 41 | T98253 | ESTs | 271 | 3.5 | 93.1 |
| 42 | W84868 | cytochrome P450, subfamily IVA, polypeptide 11 | 272 | 3.4 | 84.6 |
| 43 | H05140 | regucalcin (senescence marker protein-30) | 273 | 3.4 | 85.7 |
| 44 | H09818 | ESTs | 274 | 3.4 | 82.6 |
| 45 | W01048 | ESTs | 275 | 3.3 | 77.8 |
| 46 | AI289110 | metallothionein 1L | 276 | 3.3 | 82.8 |
| 47 | H77535 | ESTs, Weakly similar to choline kinase isolog | 277 | 3.3 | 83.3 |
| | | 384D8_3 [H. sapiens] | | | |
| 48 | T60160 | ESTs, Moderately similar to MM46 [H. sapiens] | 278 | 3.3 | 89.7 |
| 49 | H14604 | ESTs, Weakly similar to C. elegans cDNA yk30b3.5 | 279 | 3.3 | 79.2 |
| 50 | T98394 | Solute carrier family 7 (cationic amino acid | 280 | 3.3 | 85.7 |
| | | transporter, y+ system), member 7 | | | |
| 51 | H48148 | ESTs, Weakly similar to AIF-1 [H. sapiens] | 281 | 3.3 | 79.3 |
| 52 | AA453783 | Homo sapiens mRNA; cDNA DKFZp564B1264 | 282 | 3.3 | 82.1 |
| | | (from clone DKFZp564B1264) | | | |
| 53 | T69767 | hydroxyacyl-coenzyme A dehydrogenase/3-ketoacyl- | 283 | 3.2 | 84.0 |
| | | coenzyme A thiolase/enoyl-coenzyme A | | | |
| | | hydratase (trifunctional protein), β subunit | | | |
| 54 | AA446650 | Homo sapiens mRNA; cDNA DKFZp586M0723 | 284 | 3.2 | 75.0 |
| | | (from clone DKFZp586M0723) | | | |
| 55 | AA416875 | ESTs | 285 | 3.2 | 95.7 |
| 56 | R54850 | biphenyl hydrolase-like (serine hydrolase; | 286 | 3.2 | 85.7 |
| | | breast epithelial mucin-associated antigen) | | | |
| 57 | AA775957 | ATPase, Na+/K+ transporting, α 3 polypeptide | 287 | 3.2 | 93.1 |
| 58 | AA455222 | plasminogen activator, urokinase receptor | 288 | 3.2 | 77.8 |
| 59 | AA406266 | ESTs | 289 | 3.1 | 84.6 |
| 60 | N26658 | ESTs, Moderately similar to TGF-β Receptor | 290 | 3.1 | 82.1 |
| | | type III precursor [H. sapiens] | | | |
| 61 | AA455969 | prion protein (p27-30) (Creutzfeld-Jakob disease, | 291 | 3.1 | 89.7 |
| | | Gerstmann-Strausler-Scheinker syndrome, | | | |
| | | fatal familial insomnia) | | | |
| 62 | N70794 | acyl-Coenzyme A dehydrogenase, C-4 to | 292 | 3.1 | 79.3 |
| | | C-12 straight chain | | | |
| 63 | AA463454 | ESTs, [H. sapiens] | 293 | 3.1 | 82.1 |
| 64 | H23187 | carbonic anhydrase II | 294 | 3.0 | 79.3 |
| 65 | AA205598 | ESTs | 295 | 3.0 | 89.3 |
| 66 | AA670438 | ubiquitin carboxyl-terminal esterase L1 | 296 | 3.0 | 75.9 |
| | | (ubiquitin thiolesterase) | | | |
| 67 | AA121668 | pigment epithelium-derived factor | 297 | 3.0 | 86.2 |
| 68 | AA487346 | cathepsin H | 298 | 3.0 | 92.0 |
| 69 | W84701 | solute carrier family 7 (cationic amino acid | 299 | 2.9 | 77.8 |
| | | transporter, y+ system), member 8 | | | |
| 70 | AA456022 | ESTs, weakly similar to unknown [H. sapiens] | 300 | 2.9 | 81.5 |
| 71 | AA424905 | ESTs | 301 | 2.9 | 86.2 |
| 72 | W87747 | ESTs | 302 | 2.9 | 78.3 |
| 73 | R76614 | ESTs | 303 | 2.9 | 78.3 |
| 74 | AA132964 | ESTs | 304 | 2.9 | 78.6 |
| 75 | AA169798 | biphenyl hydrolase-like (serine hydrolase; | 305 | 2.8 | 86.2 |
| | | breast epithelial mucin-associated antigen) | | | |
| 76 | H15504 | Annexin A7 | 306 | 2.8 | 85.2 |
| 77 | H22856 | Glutamic-oxaloacetic transaminase 1, | 307 | 2.8 | 82.1 |
| | | soluble (aspartate aminotransferase 1) | | | |
| 78 | AA429946 | ESTs, Highly similar to peroxisomal short-chain | 308 | 2.8 | 82.8 |
| | | alcohol dehydrogenase [H. sapiens] | | | |
| 79 | R93551 | aldehyde dehydrogenase 5 | 309 | 2.8 | 85.7 |
| 80 | AA256123 | fragile histidine triad gene | 310 | 2.8 | 81.8 |
| 81 | AA621183 | solute carrier family 5 (inositol transporters), member 3 | 311 | 2.8 | 76.0 |
| 82 | R67147 | crystallin, μ | 312 | 2.8 | 76.0 |
| 83 | AA156988 | iron-responsive element binding protein 1 | 313 | 2.7 | 78.6 |
| 84 | AA459668 | 3-hydroxyisobutyryl-coenzyme A hydrolase | 314 | 2.7 | 75.9 |
| 85 | N46098 | biphenyl hydrolase-like (serine hydrolase; breast | 315 | 2.6 | 77.8 |
| | | epithelial mucin-associated antigen) | | | |
| 86 | W81371 | ESTs | 316 | 2.6 | 75.9 |
| 87 | H99883 | KIAA0828 protein | 317 | 2.6 | 79.2 |
| 88 | N65985 | ESTs | 318 | 2.6 | 75.9 |
| 89 | AA400258 | Human DNA sequence from clone 215D11, | 319 | 2.5 | 75.0 |
| | | chromosome 1p36 12-36.33 Contains a gene for | | | |
| | | a RNA-binding protein regulatory subunit, gene similar | | | |
| | | to rat gene 33, pseudogene similar to PLA-X, | | | |
| | | ESTs, STSs, GSSs and CpG islands | | | |
| 90 | AA456595 | ESTs | 320 | 2.5 | 78.3 |
| 91 | N31492 | flavin containing monooxygenase 4 | 321 | 2.5 | 75.0 |
| 92 | R28294 | glycine cleavage system protein H (aminomethyl carrier) | 322 | 2.5 | 85.7 |
| 93 | AA430382 | nucleoside phosphorylase | 323 | 2.5 | 82.8 |
| 94 | AA521401 | pyruvate dehydrogenase (lipoamide) β | 324 | 2.4 | 78.6 |
| 95 | AA453691 | Aminolevulinate, delta-, synthase 1 | 325 | 2.4 | 75.9 |
| 96 | N99256 | ESTs | 326 | 2.4 | 80.0 |
| 97 | AA448184 | ubiquinol-cytochrome c reductase, | 327 | 2.4 | 75.9 |
| | | Rieske iron-sulfur polypeptide 1 | | | |
| 98 | AA056390 | RD RNA-binding protein | 328 | 2.4 | 81.5 |
| 99 | H78368 | ESTs | 329 | 2.3 | 76.9 |
| 100 | AA432268 | ESTs | 330 | 2.3 | 75.9 |
| 101 | AA453679 | Dihydrolipoamide dehydrogenase (E3 | 331 | 2.3 | 75.9 |
| | | component of pyruvate dehydrogenase complex, | | | |
| | | 2-oxo-glutarate complex, branched chain keto | | | |
| | | acid dehydrogenase complex) | | | |

*The values in this column are the % of CC-RCC patients in whom a given gene was expressed at least 2-fold lower compared to control kidney tissue. Genes included in this Table met or exceeded this threshold in at least 75% of CC-RCC patients (but did not exceed the threshold of 3 fold down-regulation in this percentage of patients

TABLE 6A and 6B
First Set of Genes Differentially Expressed in Aggressive
vs. Non-aggressive type CC-RCC

| | | | | |
|---|---|---|---|---|
| GENBANK | | SEQ | | |
| ACCESSION # | NAME (FROM RESEARCH GENETICS) | ID NO: | CELERA # | E VALUE |

TABLE 6A
Genes upregulated in non aggressive CC-RCC

| | | | | | |
|---|---|---|---|---|---|
| 1 | N35086 | FYN oncogene related to Src, Fgr, Yes | 1 | hCG34806 | 0 |
| 2 | N66144 | | 1 | hCG34806 | 10⁻¹⁵³ |
| 3 | T47312 | Insulin receptor | 2 | hCG21793 | 10⁻¹⁸⁰ |
| 4 | AA001614 | | 2 | hCG21793 | 10⁻¹¹⁴ |
| 5 | T80232 | phosphodiesterase I/nucleotide | 3 | hCG21270 | 10⁻¹⁰⁰ |
| | | pyrophosphatase 2 (autotaxin) | | | |
| 6 | AA490694 | Hevin | 4 | hCG38543 | 4 × 10⁻⁷⁸ |
| 7 | | | 5 | | |
| 8 | AA486082 | serum/glucocorticoid regulated kinase | 6 | hCG32737 | 8 × 10⁻⁹⁹ |
| 9 | W60845 | cell division cycle 42 (GTP-binding protein) | 7 | hCG15193 | 10⁻¹³⁸ |
| 10 | N34362 | regulator of G-protein signalling 5 | 7 | hCG15193 | 0 |
| 11 | AA668470 | | 7 | hCG15193 | 0 |
| 12 | H84815 | Rab9 effector p40 | 8 | hCG29658 | 10⁻¹⁵⁵ |
| 13 | AA669136 | transcription factor 4 | 9 | hCG22018 | 10⁻¹²⁷ |
| 14 | N39240 | ESTs | 9 | hCG22018 | 0 |
| 15 | W72803 | ESTs, weakly similar to KIAA0768 protein | 10 | hCG28803 | 0.081 |
| 16 | R22412 | platelet/endothelial cell adhesion molecule | 11 | hCG40093 | 10⁻¹² |
| 17 | R56211 | platelet-derived growth factor receptor, β polypeptide | 12 | hCG16146 | 0.99 |
| 18 | H74106 | LIM binding domain 2 | 13 | hCG40704 | 0 |
| 19 | H72113 | CD34 antigen | 14 | hCG21280 | 10⁻¹⁵³ |
| 20 | AA680300 | H. sapiens clone 23698 mRNA | | | |
| 21 | R32440 | | 15 | hCG17031 | 10⁻¹¹² |
| 22 | AA777910 | H. sapiens clone 23698 mRNA | 139 | no list | |
| 23 | | | 16 | | |
| 24 | AA432292 | ESTs | 17 | hCG29296 | 0.26 |
| 25 | AA055440 | Sprouty (Drosophila) homolog 1, antagonist of FGF signal | 18 | hCG28465 | 0.012 |
| 26 | H68922 | Integrin, α1 | 19 | hCG23896 | 0.08 |
| 27 | N29914 | Endothelin receptor type B | 20 | hCG32240 | 10⁻¹¹¹ |
| 28 | | | 21 | | |

TABLE 6B
Genes downregulated in aggressive CC-RCC

| | | | | | |
|---|---|---|---|---|---|
| 29 | AA775447 | α-2-macroglobulin | 22 | hCG25215 | 7 × 10⁻⁶⁷ |
| 30 | H99415 | a kinase (PRKA) anchor protein 2 | 23 | hCG28766 | 10⁻¹⁵⁸ |
| 31 | N51499 | | 23 | hCG28766 | 0 |
| 32 | AA464644 | LIM domain only 2 (rhombotin-like 1) | 24 | hCG26502 | 0 |
| 33 | N74956 | DNA-directed RNA polymerase II B (140 kD) | 25 | hCG201171 | 3 × 10⁻⁴⁵ |
| 34 | T53298 | insulin-like growth factor binding protein 7 | 25 | hCG201171 | 5 × 10⁻¹⁵ |
| 35 | AA704965 | ESTs | 26 | hCG41053 | 0.018 |
| 36 | AA099153 | tissue inhibitor of metalloproteinase 3 (TIMP) | 27 | hCG41415 | 10⁻¹⁷⁴ |
| 37 | N95226 | KIAA0758 | 28 | hCG18763 | 0 |
| 38 | T63971 | | 28 | hCG18763 | 10⁻¹²¹ |
| 39 | AA189106 | KIAA1102 | 29 | hCG33090 | 0 |
| 40 | R23270 | | 29 | hCG33090 | 10⁻¹⁰⁴ |
| 41 | N36136 | ESTs, moderately similar to endomucin | 30 | hCG39439 | 0 |
| 42 | N93505 | Transmembrane 4 superfamily member 2 | 31 | hCG18324 | 10⁻¹⁶⁰ |
| 43 | AA173408 | ESTs | 32 | hCG37431 | 10⁻¹⁰⁹ |
| 44 | T71976 | Phosphatidic acid phosphatase type 2b | 33 | hCG32470 | 0 |
| 45 | T72119 | | 33 | hCG32470 | 10⁻¹⁵⁴ |
| 46 | AA487034 | Transforming growth factor β receptor II (70-80 kD) | 34 | hCG26855 | 10⁻¹²⁷ |
| 47 | W68396 | KIAA0096 | 35 | hCG26802 | 10⁻¹²⁷ |
| 48 | N57594 | H. sapiens mRNA; cDNA DKFZp564E153 | 36 | hCG41872 | 0.98 |
| 49 | N94344 | H. sapiens mRNA; cDNA DKFZp564E153 | 37 | hCG15077 | 0.005 |
| 50 | W47641 | H. sapiens mRNA; cDNA DKFZp564E153 | 38 | hCG25175 | 1.4 |
| 51 | AA458653 | H. sapiens mRNA for GS3955, complete cds | 39 | hCG15902 | 2 × 10⁻⁹⁴ |

TABLE 7
Second Set of Genes (166) Differentially Expressed in Aggressive vs. Non-aggressive type CC-RCC

| | GENBANK | | SEQ |
|---|---|---|---|
| | ACCESSION # | NAME | ID NO: |
| 1 | H23081 | Zinc finger protein 264 | 332 |
| 2 | N25425 | v-raf-1 murine leukemia viral oncogene homologue 1 | 333 |
| 3 | AA670438 | Ubiquitin carboxyl-terminal esterase L1 (ubiquitin thiolesterase) | 334 |
| 4 | AA453273 | U6 snRNA-associated Sm-like protein | 335 |
| 5 | AA405748 | U2 small nuclear ribonucleoprotein auxiliary factor (65 kD) | 336 |
| 6 | AA432062 | Tyrosine kinase with immunoglobulin and epidermal growth factor homology domains | 337 |
| 7 | R06309 | Tumor protein D52-like 2 | 338 |
| 8 | R36467 | Transforming growth factor, β 1 | 339 |
| 9 | H50377 | Tight junction protein 1 (zona occludens 1) | 340 |
| 10 | AA778098 | Thymidine kinase 1, soluble | 341 |
| 11 | H05577 | Splicing factor 30, survival of motor neuron-related | 342 |
| 12 | AA018591 | Spectrin, β, non-erythrocytic 1 | 343 |
| 13 | R66139 | Small inducible cytokine subfamily D (Cys-X3-Cys), member 1 (fractalkine, neurotactin) | 344 |
| 14 | R96668 | Small inducible cytokine subfamily A (Cys-Cys), member 14 | 345 |
| 15 | N64837 | SFRS protein kinase 1 | 346 |
| 16 | AA070226 | Selenoprotein P, plasma, 1 | 347 |
| 17 | W96107 | Sec61 γ | 348 |
| 18 | R55105 | Sarcoglycan, β (43 kD dystrophin-associated glycoprotein)″ | 349 |
| 19 | N93715 | Ribosomal protein S29 | 350 |
| 20 | AA991856 | Ribophorin II | 351 |
| 21 | AA479781 | Radixin | 352 |
| 22 | AA464152 | Quiescin Q6 | 353 |
| 23 | AA151249 | Protoporphyrinogen oxidase | 354 |
| 24 | R79082 | protein tyrosine phosphatase, receptor type, K | 355 |
| 25 | AI022531 | protein tyrosine phosphatase, receptor type, β polypeptide | 356 |
| 26 | AA490696 | protein phosphatase 2 (formerly 2A), catalytic subunit, β isoform | 357 |
| 27 | AA916327 | protective protein for β-galactosidase (galactosialidosis) | 358 |
| 28 | AA455193 | proteasome (prosome, macropain) 26S subunit, non-ATPase, 2 | 359 |
| 29 | AA426212 | procollagen-proline, 2-oxoglutarate 4-dioxygenase (proline 4-hydroxylase), β polypeptide (protein disulfide | 360 |
| | | isomerase; thyroid hormone binding protein p55) | |
| 30 | AA488432 | phosphoserine phosphatase | 361 |
| 31 | AA402874 | phospholipid transfer protein | 362 |
| 32 | AA699876 | phosphoinositide-3-kinase, class 2, β polypeptide | 363 |
| 33 | AA629987 | peptidylprolyl isomerase D (cyclophilin D) | 364 |
| 34 | AA488969 | PDZ domain containing guanine nucleotide exchange factor(GEF)1; RA(Ras/Rap1A-associating)-GEF | 365 |
| 35 | AA845432 | parathyroid hormone-like hormone | 366 |
| 36 | AA446301 | paraoxonase 2 | 367 |
| 37 | AA451781 | novel RGD-containing protein | 368 |
| 38 | H83225 | Novel gene on chromosome 20 | 369 |
| 39 | AA709414 | nidogen (enactin) | 370 |
| 40 | N30706 | neuralized (Drosophila)-like | 371 |
| 41 | AA491124 | NAD(P)H menadione oxidoreductase 2, dioxin-inducible | 372 |
| 42 | R44617 | MyoD family inhibitor | 373 |
| 43 | R59167 | Meis (mouse) homolog 2 | 374 |
| 44 | AA155913 | matrix Gla protein | 375 |
| 45 | R39273 | MAD (mothers against decapentaplegic, Drosophila) homolog 4 | 376 |
| 46 | AA668531 | leucocyte vacuolar protein sorting 45 | 377 |
| 47 | AA459106 | kinectin 1 (kinesin receptor) | 378 |
| 48 | N70078 | KIAA1058 protein | 379 |
| 49 | H60026 | KIAA0745 protein | 380 |
| 50 | AA455507 | KIAA0618 gene product | 381 |
| 51 | AA702698 | KIAA0414 protein | 382 |
| 52 | AA284634 | Janus kinase 1 (a protein tyrosine kinase) | 383 |
| 53 | R70685 | Jagged1 (Alagille syndrome) | 384 |
| 54 | AA683550 | interleukin-1 receptor-associated kinase 1 | 385 |
| 55 | AA148200 | integrin-linked kinase | 386 |
| 56 | N74131 | Human secretory protein (P1.B) mRNA, complete cds″ | 387 |
| 57 | AA487681 | Human mRNA for ornithine decarboxylase antizyme, ORF 1 and ORF 2 | 388 |
| 58 | AA931758 | Human G0S2 protein gene, complete cds | 389 |
| 59 | AA418914 | Human DNA sequence from clone 30M3 on chromosome 6p22.1-22.3. Contains three novel genes, one similar | 390 |
| | | to C. elegans Y63D3A.4 and one similar to (predicted) plant, worm, yeast and archaea bacterial genes, and the | |
| | | first exon of the KIAA0319 gene. Co | |
| 60 | AA480820 | Human 1.1 kb mRNA upregulated in retinoic acid treated HL-60 neutrophilic cells | 391 |
| 61 | AA401736 | Human ubiquitously-expressed transcript (UXT) mRNA | 392 |
| 62 | N73309 | Human signal sequence receptor, γ (translocon-associated protein γ (SSR3), mRNA | 393 |
| 63 | T70352 | Homo sapiens mRNA; cDNA DKFZp564O222 (from clone DKFZp564O222) | 394 |
| 64 | AA664020 | Homo sapiens mRNA; cDNA DKFZp564M0763 (from clone DKFZp564M0763) | 395 |
| 65 | N55339 | Homo sapiens mRNA; cDNA DKFZp564H1916 (from clone DKFZp564H1916) | 396 |
| 66 | N58145 | Homo sapiens lipoma HMGlC fusion partner (LHFP) mRNA | 397 |
| 67 | N27165 | Homo sapiens clone 24582 mRNA sequence | 398 |
| 68 | T98002 | Homo sapiens chromosome 19, cosmid F22329 | 399 |
| 69 | AA700688 | Homo sapiens ATP synthase, H+ transporting, mitochondrial F1 complex, ε subunit (ATP5E), nuclear gene | 400 |
| | | encoding mitochondrial protein, mRNA | |
| 70 | T41173 | Homo sapiens a disintegrin-like and metalloprotease (reprolysin type) with thrombospondin type 1 motif, 1 | 401 |
| | | (ADAMTS1), mRNA | |
| 71 | AA873089 | H. sapiens DNA for cyp related pseudogene | 402 |
| 72 | AA487912 | guanine nucleotide binding protein (G protein), β polypeptide 1 | 403 |
| 73 | AA629909 | glycyl-tRNA synthetase | 404 |
| 74 | AA122287 | glycoprotein A repetitions predominant | 405 |
| 75 | AA152347 | glutathione S-transferase A4 | 406 |
| 76 | AA444009 | glucosidase, α; acid (Pompe disease, glycogen storage disease type II) | 407 |
| 77 | AA878899 | galactosidase, β 1 | 408 |
| 78 | N22980 | FYN oncogene related to SRC, FGR, YES | 409 |
| 79 | AA865707 | fibrinogen, A α polypeptide | 410 |
| 80 | AA679352 | farnesyl-diphosphate farnesyltransferase 1 | 411 |
| 81 | AA677650 | ESTs, Weakly similar to similar to coiled-coil protein [C. elegans] | 412 |
| 82 | AA464143 | ESTs, Weakly similar to RNA polymerase II elongation factor ELL2 [H. sapiens] | 413 |
| 83 | AA098892 | ESTs, Weakly similar to R12E2.12 [C. elegans] | 414 |
| 84 | N76361 | ESTs, Weakly similar to putative Rho/Rac guanine nucleotide exchange factor [H. sapiens] | 415 |
| 85 | AA778640 | ESTs, Weakly similar to leucine aminopeptidase [H. sapiens] | 416 |
| 86 | AA488171 | ESTs, Weakly similar to formin 4 [M. musculus] | 417 |
| 87 | AA449345 | ESTs, Weakly similar to F48E8.2 [C. elegans] | 418 |
| 88 | W73797 | ESTs, Weakly similar to Containing ATP/GTP-binding site motif A (P-loop) similar to C. elegans | 419 |
| | | protein(P1: CEC47E128); similar to mouse α-mannosidase(P1: B54407) [H. sapiens] | |
| 89 | R67283 | ESTs, [H. sapiens] | 420 |
| 90 | AA706829 | ESTs, Moderately similar to putative Rab5-interacting protein {clone L1-57} [H. sapiens] | 421 |
| 91 | AA446651 | ESTs, Moderately similar to Kryn [M. musculus] | 422 |
| 92 | R16957 | ESTs, Highly similar to Jκ recombination signal binding protein [H. sapiens] | 423 |
| 93 | AA149204 | ESTs, Highly similar to growth arrest inducible gene product [H. sapiens] | 424 |
| 94 | H73484 | ESTs, Highly similar to CGI-106 protein [H. sapiens] | 425 |
| 95 | AA011593 | ESTs, Highly similar to cell adhesion regulator [R. norvegicus] | 426 |
| 96 | AA460005 | ESTs, Highly similar to antigen NY-CO-33 [H. sapiens] | 427 |
| 97 | AA416627 | ESTs | 428 |
| 98 | N80361 | ESTs | 429 |
| 99 | AA279648 | ESTs | 430 |
| 100 | N49774 | ESTs | 431 |
| 101 | R45672 | ESTs | 432 |
| 102 | AA463189 | ESTs | 433 |
| 103 | AA418040 | ESTs | 434 |
| 104 | N34799 | ESTs | 435 |
| 105 | AA030013 | ESTs | 436 |
| 106 | N36098 | ESTs | 437 |
| 107 | N72847 | ESTs | 438 |
| 108 | R16545 | ESTs | 439 |
| 109 | R91821 | ESTs | 440 |
| 110 | AA101971 | ESTs | 441 |
| 111 | T52564 | ESTs | 442 |
| 112 | AA455962 | ESTs | 443 |
| 113 | W69216 | ESTs | 444 |
| 114 | N66734 | ESTs | 445 |
| 115 | N89738 | ESTs | 446 |
| 116 | W84486 | ESTs | 447 |
| 117 | AA464603 | ESTs | 448 |
| 118 | W31683 | ESTs | 449 |
| 119 | AA126673 | ESTs | 450 |
| 120 | AA664044 | ESTs | 451 |
| 121 | AA149640 | ESTs | 452 |
| 122 | AA436184 | ESTs | 453 |
| 123 | N24042 | ESTs | 454 |
| 124 | AA290631 | ESTs | 455 |
| 125 | AA150505 | ESTs | 456 |
| 126 | AA009755 | ESTs | 457 |
| 127 | AA452118 | ESTs | 458 |
| 128 | AA452165 | ESTs | 459 |
| 129 | N32226 | ESTs | 460 |
| 130 | N92478 | ESTs | 461 |
| 131 | N46849 | ESTs | 462 |
| 132 | AA460463 | ESTs | 463 |
| 133 | T95200 | ESTs | 464 |
| 134 | AA454008 | ESTs | 465 |
| 135 | H99766 | ESTs | 466 |
| 136 | AA191322 | ESTs | 467 |
| 137 | AA701521 | ESTs | 468 |
| 138 | H41160 | ESTs | 469 |
| 139 | AA455094 | ESTs | 470 |
| 140 | AA284245 | ESTs | 471 |
| 141 | AA197344 | ESTs | 472 |
| 142 | R62868 | erythrocyte membrane protein band 7.2 (stomatin) | 473 |
| 143 | AA052960 | Dyskeratosis congenita 1, dyskerin | 474 |
| 144 | AA621202 | DKFZP586D1519 protein | 475 |
| 145 | AA496996 | DKFZP564F0522 protein | 476 |
| 146 | AA447502 | DKFZP564B147 protein | 477 |
| 147 | AA704226 | DKFZP434G162 protein | 478 |
| 148 | R07560 | Deoxyguanosine kinase | 479 |
| 149 | AA629999 | Cytochrome c oxidase subunit VIIb | 480 |
| 150 | AA486312 | Cyclin-dependent kinase 4 | 481 |
| 151 | AA292226 | Creatine transporter [human, brainstem/spinal cord, mRNA, 2283 nt] | 482 |
| 152 | AA150402 | Collagen, type IV, α 1 | 483 |
| 153 | AA476282 | Coated vesicle membrane protein | 484 |
| 154 | W81562 | Cell division cycle 42 (GTP-binding protein, 25 kD) | 485 |
| 155 | W81563 | Cell division cycle 42 (GTP-binding protein, 25 kD) | 486 |
| 156 | AA043228 | Calponin 3, acidic | 487 |
| 157 | R76554 | Calmodulin 1 (phosphorylase kinase, delta) | 488 |
| 158 | AA487552 | Calcium binding atopy-related autoantigen 1 | 489 |
| 159 | AA757351 | Calcitonin receptor-like | 490 |
| 160 | AA456480 | BCL2-like 2 | 491 |
| 161 | H14372 | ATP-binding cassette, sub-family A (ABC1), member 5 | 492 |
| 162 | H38623 | ATP synthase, H+ transporting, mitochondrial F0 complex, subunit f, isoform 2 | 493 |
| 163 | AA046701 | ATP synthase, H+ transporting, mitochondrial F0 complex, subunit c (subunit 9), isoform 1 | 494 |
| 164 | AA633658 | Amyloid β (A4) precursor protein (protease nexin-II, Alzheimer disease) | 495 |
| 165 | W42849 | Amyloid β (A4) precursor protein (protease nexin-II, Alzheimer disease) | 496 |
| 166 | N72918 | Adaptor-related protein complex 2, β 1 subunit | 497 |

### EXAMPLE IV

### Molecular Heterogeneity in CC-RCC

Having identified common alterations in gene expression in CC-RCC tissue, the inventors next sought to identify DNA expression patterns that account for the heterogeneity in the clinical behavior of the disease. Some of the tumors were highly aggressive, leading to patients' deaths within three years, while other patients had no recurrences following surgery (Table 1). The present inventors thus sought to discover gene expression signatures that could identify, predict and possibly account for the lethal tumor phenotype.

A number of methods have been employed to identify DNA expression profiles that were correlated with some observable phenotype or property of cells or tissue. Ahizadeh et al. (supra) performed hierarchical clustering and then searched for selectively expressed groups, while Golub et al. (supra) and others ranked individual DNAs based on their ability to classify patients (Science (2000) 286:531-7). As discussed in Hastie et al., (GenomeBiology.com (2001) 2, RESEARCH0003), the present strategy called for, first, clustering the DNAs and then assessing the subclusters' ability to differentiate patients. This approach allows exploitation of the value of correlated sets of DNAs and takes advantage of a systematic, mathematical test. The present inventors also performed individual DNA permutation analysis to generate statistical significance values for the ability to make a classification based on an individual DNA.

The inventors compared the expression profiles using a selected set of 3,184 polynucleotides that registered expression ratios greater than 2 (up-or down-regulated) in at least 2 tumors (where results were consistently present in at least 75% of the experiments).

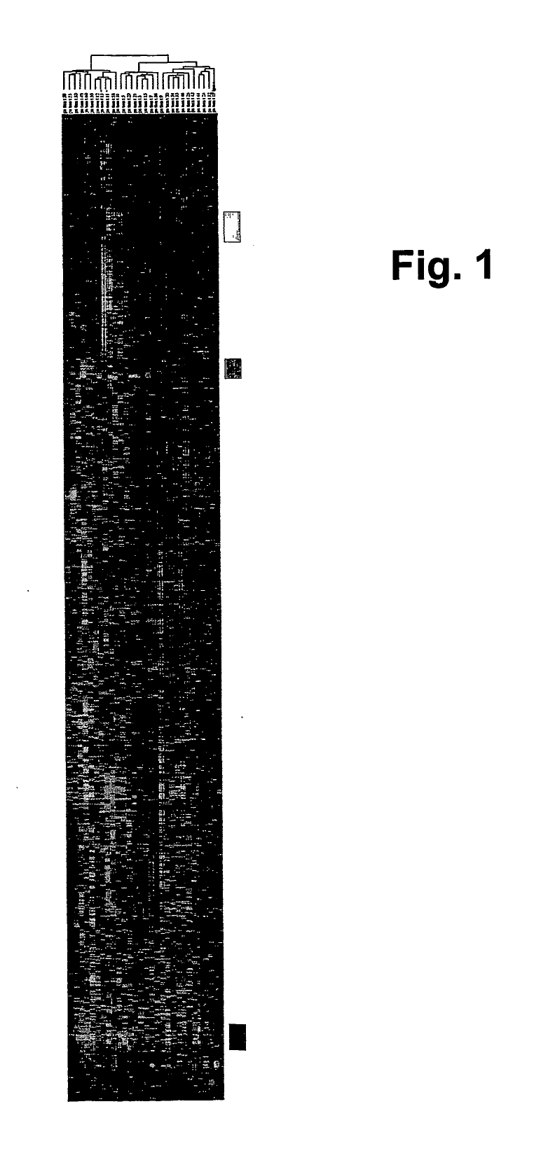

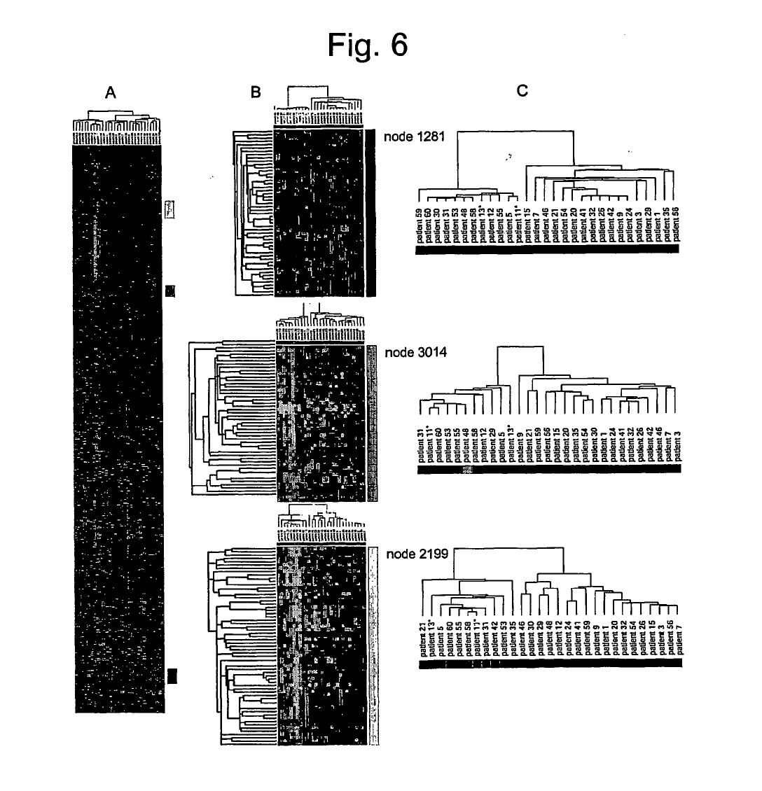

The data was median polished, organized, and visualized using average-linkage hierarchical clustering (Eisen, M B. et al., (1998) Proc Natl Acad Sci USA 95:14863-4868) (FIG. 6A/FIG. 1). This method arranges DNAs and patients according to similarity in pattern of expression. Many distinct trends in expression were identified by organization of the color patterns in the matrix. However, visual discernment of which clusters are most relevant biologically and clinically was cumbersome.

To circumvent a manual investigation of the correlation between each sub-cluster of DNAs and each clinical parameter, the inventors implemented the program CLUSTERFINDER described above. This program scores and identifies groups of clustered DNAs (nodes in the dendrogram) that best differentiate patients based on a known clinical distinction. The analysis was biased toward highly correlated DNA clusters by scoring only clusters with >10 DNAs and correlation coefficients >0.5.

The inventors tested two clinical parameters corresponding to two hypotheses of tumor progression. First, “tumor staging” was used as the discriminating clinical parameter, under the assumption that gene expression profiles change as a tumor progresses. The tumors were divided into two groups: (1) stage I and II and (2) stage III and IV. Surprisingly, this distinction did not correlate strongly with any subclusters within the DNA expression matrix.

Second, the inventors used “patient outcome” as the discriminating parameter, under the hypothesis that multiple classes of CC-RCC exist, each having a distinct molecular profile that would correspond to clinical course. For this operation, the inventors distinguished between those patients that died due to cancer within 5 years of initial diagnosis, and those that survived cancer-free for >5 years (Table 1). Also included in the “poor outcome” class were two patients who survived with cancer for 89.4 and 105.6 months. For this “patient outcome” parameter, multiple clusters of DNAs distinguished classes of patients. Cluster 687, containing 24 DNAs, and its parent, Cluster 1281, containing 51 DNAs, had the highest predictive scores (1.70). Cluster 3014, with 48 DNAs, and cluster 2199, with 61 DNAs, also had strong predictive scores (1.46, 1.011).

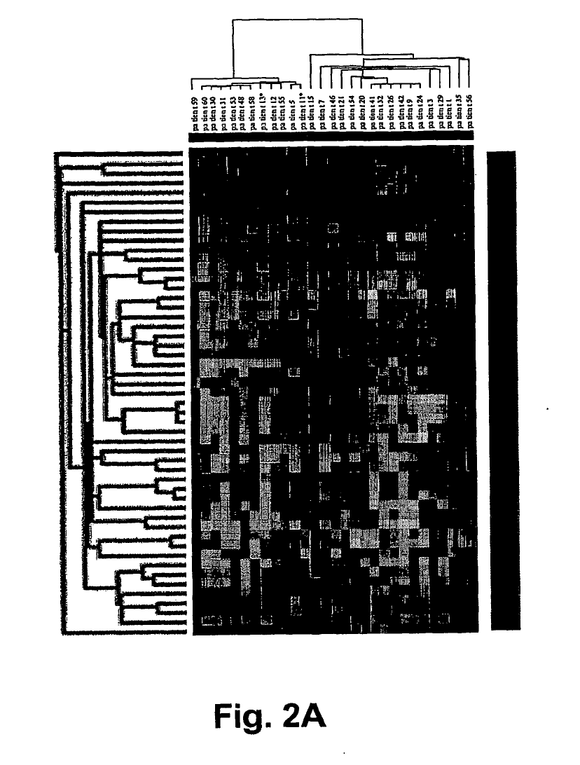

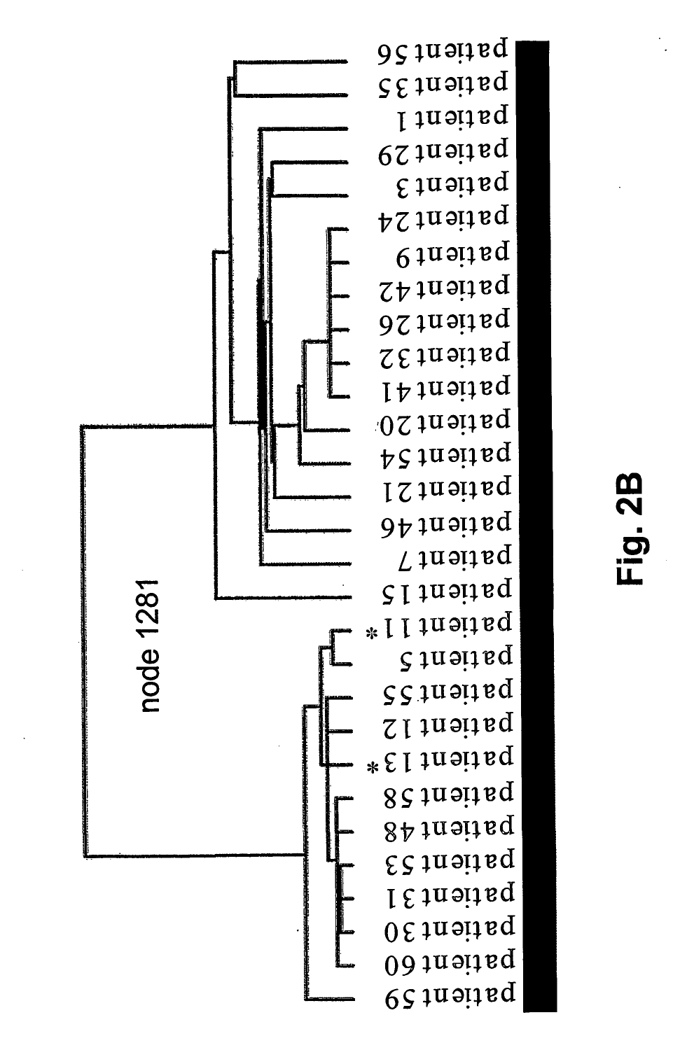

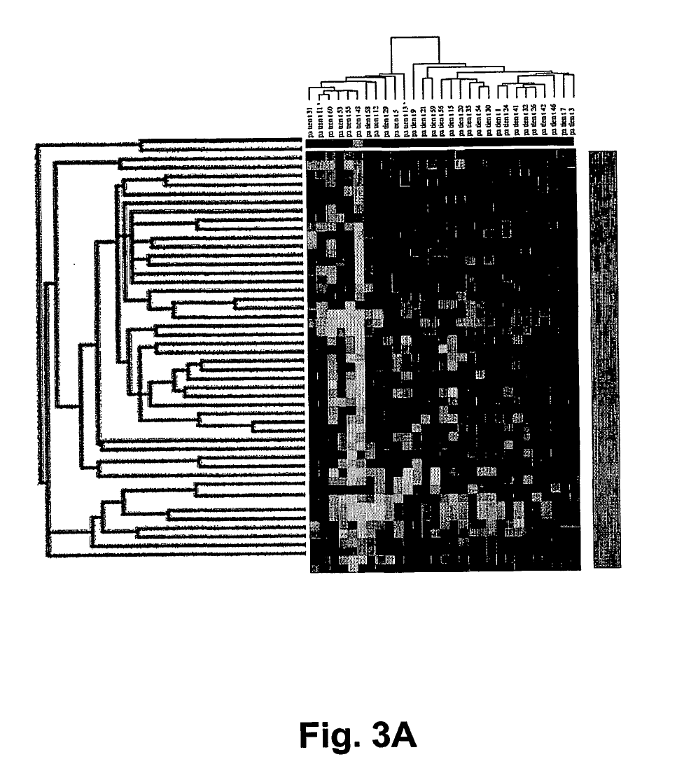

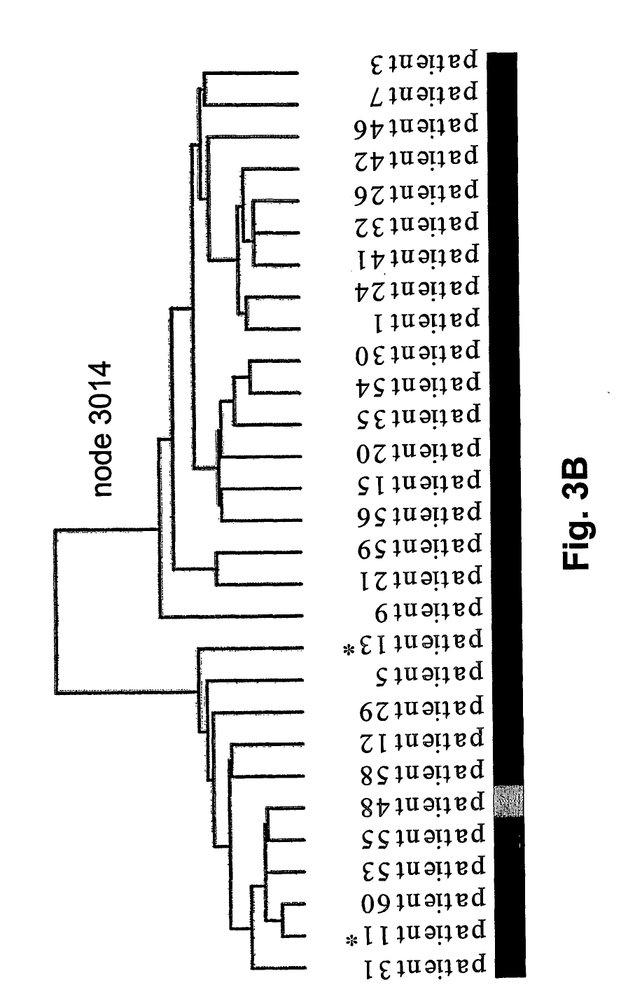

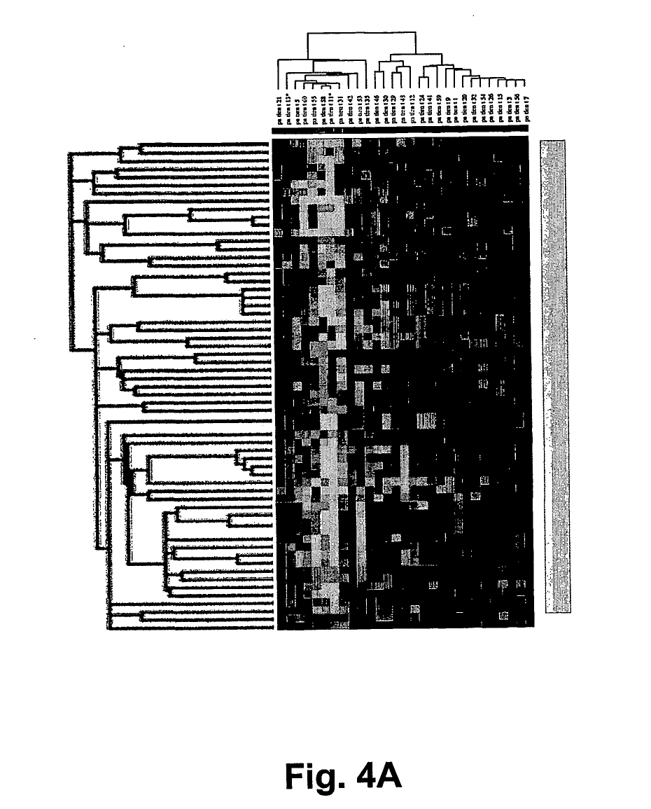

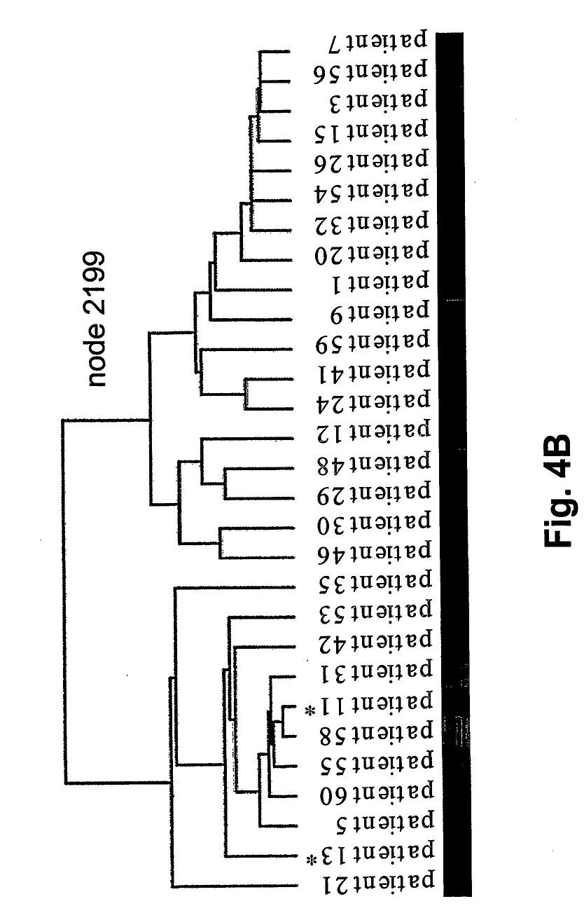

FIGS. 6B, 6C and FIGS. 2A, 2B, 3A, 3B, 4A and 4B depict the re-clustering of patients based on these subclusters. Cluster 1281 displays marked separation of the two classes of patients, with the exception of patient 30. Cluster 3014 also separates the patients well, although expression values within this cluster did not correlate as highly.

The significance of this underlying molecular profile was confirmed using a modified permutation t-test. 217 DNAs differentiated the two outcome groups significantly (α<0.001). All 51 DNAs within Cluster 1281 (see also Table 6) were present in this group of 217 DNAs (Table 6 +Table 7).

Thus, Table 6 shows the 51 sequences of greatest interest in their ability to distinguish between the two clinical types of CC-RCC discerned by the present inventors: aggressive and non-aggressive. Table 6A shows 28 genes (SEQ ID NO:1-21 and SEQ ID NO:139) whose expression is upregulated non-aggressive cases of CC-RCC (tumor compared to normal tissue). In contrast, Table 6B lists 23 genes (apparently 19 unique sequences designated SEQ ID NO:22-39) that are down-regulated in aggressive CC-RCC (tumor tissue relative to normal kidney tissue). On the basis of these expression patterns of as few as 1 gene and as many as all 51 gene probes (apparently 39 or 40 unique sequences; SEQ ID NO:1-39 and SEQ ID NO:139), it is possible to obtain a molecular classification of CC-RCC into the two clinically distinct classes. This serves as the basis of an routine molecular prognostic assay that can be done to classify CC-RCC patients and tailor their therapy and follow-up programs in accordance with their prognosis.

### EXAMPLE V

### Clinical Simulation

These discriminating clusters of DNAs have at least two applications: providing insight into potential molecular subtypes of CC-RCC, and as a means for objective and accurate determination of patient prognosis. To address the second, the present inventors performed a clinical simulation. Because the DNAs in these identified clusters were ordered using the molecular profiles of the 29 patients, testing the predictive ability of these DNAs on the same 29 patients would be biased. To remove this bias, each patient's data was systematically treated as if it came from an unknown test patient who had just undergone nephrectomy and a molecular profile screening with the present cDNA probe set, while the remaining 28 patients served to populate the database of known molecular profiles/clinical follow-up data. The same analysis protocol described above was followed independently of the test patient.

A flow diagram of the simulation process is shown below.

By sequential removal of any individual patient (being treated as an “unknown”) from the clusters, the clustering of DNAs was slightly altered so that the clusters were no longer identical in structure to the originally predictive clusters. Throughout this simulation, the set of DNAs identified as Cluster 1281 consistently clustered together, as expected from their high correlation index in the original clustering operation. Although a few DNAs appeared in this grouping sporadically, on average, 95% of the DNAs in the original cluster were also present in the simulation clusters identified by CLUSTERFINDER However, DNAs in the other previously identified clusters did not maintain their order during the simulation. This follows from the fact that these other clusters did not have as high correlation indices in the original operation.

Since the clusters containing DNAs similar to Cluster 1281 maintained high predictive scores and consistent DNA content throughout the simulation, the inventors used these as predictive tests for the respective “test” patients. The test patient's prognosis was predicted by comparing his profile with that of the independently established predictive cluster (i.e., from which the “test” patient's data had been removed).

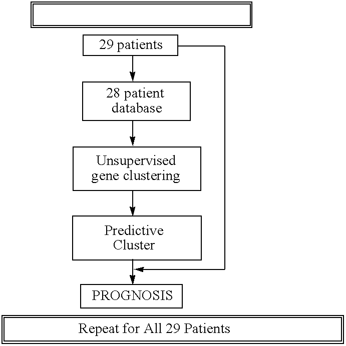

Remarkably, the clusters of DNAs similar to those in Cluster 1281 independently permitted correct prediction of patient outcomes correctly in all but one case. This one prediction failure was a patient with advanced stage cancer who survived >5 years. The test never failed to predict patients with poor outcomes. The comparison of patient prognosis based on staging vs. molecular profiling is presented in the two rightmost columns of Table 1.

### EXAMPLE VI

### Content of DNAs in the Predictive Cluster

Since the DNAs within Cluster 1281 proved predictive and stable throughout the simulation, the inventors investigated the DNAs within this cluster and their potential implication in the biology of the highly aggressive sub-type of CC-RCC.

Tables 2 and 3 present a subset of 123 genes that are generally up-regulated in CC-RCC tissue versus normal kidney tissue. Table 2 shows the most consistently and/or strongly upregulated “first” (most preferred) subset of genes (SEQ ID NO: 40-68). These genes are up-regulated at least 3-fold in 75% or more of the CC-RCC patients. Table 3 shows a second set of 91 up-regulated genes (SEQ ID NO:140-230) which are up-regulated at least 2-fold in 75% or more of the CC-RCC patients.

Tables 4 and 5 present a subset of 178 genes that are down-regulated in CC-RCC tissue versus normal kidney tissue. Table 4 shows the 77 most consistently and/or strongly down-regulated “first” (most preferred) subset of genes (SEQ ID NO:69-138). These genes are down-regulated by at least 3-fold in 75% or more of the CC-RCC patients. Table 5 shows a second set of 101 down-regulated genes (SEQ ID NO:231-331) that are down-regulated by at least 2-fold in 75% or more of the CC-RCC patients.

The gene products (taken from serum, urine, saliva, or other abundant body fluid rather than kidney tissue) of the up-regulated expressed nucleic acids (Tables 2 and 3) can be assayed using in immunoassays known in the art (i.e., ELISA, immunocytochemistry, sandwich assays, etc.) for the purpose of diagnosing patients with CC-RCC but do not discriminating between the heterogeneous disease severity.

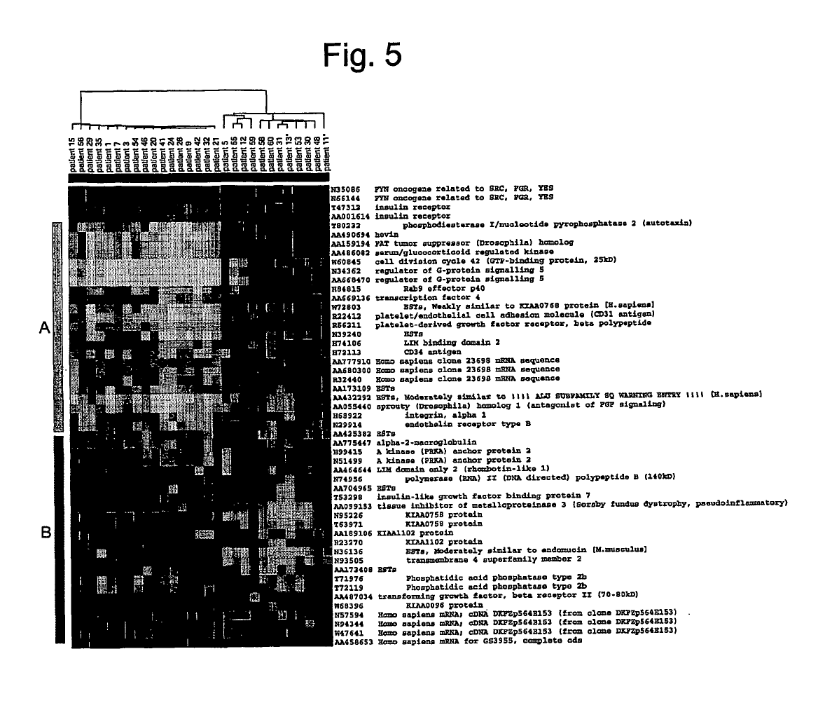

Differentially expressed nucleic acids indicative of aggressive versus non-aggressive disease phenotype are not included in this subset but were independently determined by the inventors through clustering and t-statistics. The list of expressed nucleic acids discovered to be indicative are summarized in FIG. 6A and Tables 6 and 7. FIG. 5 represents the actual relative expression values for the 51 cDNAs that comprise Cluster 1281. (See also Table 6.) The inventors have shown that these 51cDNAs are down regulated in the aggressive CC-RCC phenotype (SEQ ID NO:22-39) or up-regulated in non-aggressive CC-RCC (SEQ ID NO: 1-21 and 139). A larger set of genes that are differentially expressed in aggressive vs. non-aggressive CC-RCC includes the 166 probes SEQ ID NO:332-497, inclusive. It is not yet clear how these genes break down into the two categories so far identified in these prognostic genes.

These two classes of gene can be viewed as
(A) positive effectors of less aggressive CC-RCC; and
(13) inhibitors of tumor progression that would keep less aggressive CC-RCC in check.
   For example PDGFR expression was said to be an indicator of proliferation in other cancers (Lafuente, et al. (1999) J Mol Neurosci 13:177-85) while sprouty homologue 1 (D. melanogaster) negatively modulates angiogenesis by inhibiting tyrosine kinase-mediated signaling pathways (Lee, S H et al. (2000) J Biol Chem 26:26) such as the VEGF pathway. It is noteworthy that VEGF was highly up regulated in all CC-RCC cases tested.

The DNAs in Group II are almost exclusively down regulated in the highly aggressive CC-RCC cases. This group includes TGFβRII, TIMP3, and insulin-like growth factor binding protein 7 (IGF-BP7). All of these genes/proteins have been implicated in late-stage or aggressive cancer.

### EXAMPLE VII

### Expression of Specific Genes in CC-RCC

Ceruloplasmin, a protein involved in iron and copper homeostasis, had the highest increase in expression in CC-RCC vs. normal tissue. Interestingly, only a handful of reports showed an association between ceruloplasmin and CC-RCC. One study reported secretion of this protein by CC-RCC (Saito, K., et al. (1985) Biochem Med 33:45-52) and the other reported its elevation in RCC patient serum (Pejovic, M. et al. (1997) Int Urol Nephrol 29:427-32). The present discovery merits an in-depth investigation of ceruloplasmin's role in CC-RCC tumorigenesis and its potential value as a tumor marker.

Another copper-related protein, lysyl oxidase (1 1-fold up-regulated in 95% of CC-RCC) is an extracellular enzyme involved in connective tissue maturation. It is highly expressed in invasive breast cancer cell lines (Kirschmann, D A et al., (1999) Breast Cancer Res Treat 55:127-136) but has never been studied in RCC.

Finally, a well-known angiogenic factor, VEGF, has been shown to be highly expressed in RCC (Takahashi, A et al., (1994) Cancer Res 54:4233-4237; Thelen, P et al., (1999) Anticancer Res 19:1563-1565) and elevated in the serum of RCC patients (Sato, K et al., (1999) Jpn J Cancer Res 90:874-879; Wechsel, H W et al., (1999) Anticancer Res 19:1537-540). The present invention corroborated those observations and showed an average 5-fold up-regulation of VEGF in 96% of the CC-RCC tunors.

The present invention also identified a large number of examples of prominent down-regulation of DNAs in CC-RCC. Most strikingly, kininogen was more than 27-fold down-regulated. This protein, involved in the activation of the clotting system, has recently been shown to be anti-angiogenic (Zhang, J C et al. (2000) FASEB J 14: 2589-600). Its down regulation, never before reported in CC-RCC, in combination with the up-regulation of VEGF might explain the characteristic hypervascularization of CC-RCC.

The metallothionein (AT) family of genes was coordinately down regulated in CC-RCC. The products of these genes modulate the release of hydroxyl radicals and the exchange of heavy metals such as zinc, cadmium or copper. Differential expression of this class of genes has been reported in many cancers (Janssen, et al., (2000) J Pathol 192:293-300). Several subtypes, MT-1A, MT-1G, MT-1H were reported to be down regulated in RCC (Nguyen et al., (2000) Cancer Lett 160:13340; Izawa, et al., (1998) Urology 52:767-72). The present invention supports these reports and adds the fact that MT-1L and MT-1E were down-regulated.

Based on this model, the present inventors conceived that a distinctive molecular profile exists early in tumor development. The more aggressive type tumor progresses much more rapidly, and thus usually presents clinically at a more advanced stage, while tumors of the less aggressive class progress slowly and usually present clinically before tumor cells have invaded sites outside the kidney. This model is strongly supported by the dataset disclosed herein. Indeed, only one patient with CC-RCC having the aggressive molecular signature survived >5 years. This patient presented with stage m cancer, but 7 years later, had no evidence of disease (“NED”; Patient 30, Table 1).

Remarkably, the “molecular signature” approach of the present invention was of sufficient robustness to predict correctly the outcome in five cases in which the clinico-pathological information would have suggested otherwise.

One patient with the non-aggressive molecular signature had, at surgery, a grade 3 tumor invading the renal vein, but has since survived for 7.5 years (Patient 29, Table 1).

Another patient, with a stage II, grade 2 tumor went on to die of the cancer 4.6 years after surgery (Patient 55, Table 1 and Figures). Using the present molecular signature, the latter patient was classified as having the aggressive subtype.

Another patient with bone metastasis at diagnosis is still alive after 8.8 years and survives despite the bone metastasis, which is stable (Patient 54, Table 1 and Figures). Using the present approach, this patient was identified as having the non-aggressive molecular signature.

These cases and others demonstrate that the set of genes described herein, or a subset thereof, is useful in determining the prognosis of patients with CC-RCC.

Loss of the TGFβII signaling pathway in late stages of RCC has previously been shown.

TIMP3 is known to be downstream of TGFβ and is a known tumor suppressor gene. By inhibiting the function of matrix metalloproteinases, TIMP3 regulates cell adhesion and extracellular matrix homeostasis. Loss of TIMP3 expression by promoter methylation was shown to increase tumorigenicity due to unregulated MMPs (Bachman, et al., (1999) Cancer Res 59:798-802).

The present out clustering methodology has effectively demonstrated correlation of an entire pathway and its exclusive down regulation in the aggressive cancers. The ligands, the receptors and a downstream effectors are all down regulated and all are implicated in aggressive cancer.

The references cited above are all incorporated by reference herein, whether specifically incorporated or not.

1-42. (canceled)

43. A method of predicting whether a clear cell renal cell carcinoma (CC-RCC) in a subject is of a non-aggressive or aggressive type, comprising:
(a) examining the expression in tumor tissue from the subject of nucleic acid that hybridizes at high stringency conditions with
(i) one or more oligonucleotide or polynucleotide probes having the sequence of SEQ ID NO:1 through SEQ ID NO:21, inclusive; and/or
(ii) one or more oligonucleotide or polynucleotide probes having the sequence of SEQ ID NO: 22 through SEQ ID NO:39 inclusive;
(b) examining the expression in normal kidney tissue of the subject of nucleic acid that hybridizes at high stringency conditions with the oligonucleotide or polynucleotide probes of (a)(i) or (a)(ii);
(c) comparing the expression in tumor tissue in step (a) with the expression in normal tissue in step (b),
wherein:
(1) when the expression of nucleic acids hybridizing to the probe or probes of (a)(i) is up-regulated at least 2-fold in the tumor tissue compared to the normal kidney tissue, the CC-RCC is predicted to be non-aggressive; and/or
(2) when the expression of nucleic acids hybridizing to the probe or probes of (a)(ii) is down-regulated at least 2-fold, in the tumor tissue compared to the normal kidney tissue, the CC-RCC is predicted to be aggressive.

44. The method of claim 43, wherein, when the expression of the nucleic acids hybridizing to the probe or probes of (a)(i) is up-regulated at least 3-fold in the tumor tissue compared to the normal kidney tissue, the CC-RCC is predicted to be non-aggressive.

45. The method of claim 44, wherein, when said expression is up-regulated at least 4-fold in the tumor tissue compared to the normal kidney tissue, the CC-RCC is predicted to be non-aggressive.

46. The method of claim 43, wherein, when the expression of nucleic acids hybridizing to the probe or probes of (a)(ii) is down-regulated by at least 3-fold, in the tumor tissue compared to the normal kidney tissue, the CC-RCC is predicted to be aggressive.

47. The method of claim 46, wherein, when said expression is down-regulated by at least 4-fold, in the tumor tissue compared to the normal kidney tissue, the CC-RCC is predicted to be aggressive.

48. The method of claim 43 wherein the expression is examined with probes having the sequence of SEQ ID NO: 1 through SEQ ID NO:21 inclusive.

49. The method of claim 43 wherein the expression is examined with probes having the sequence of SEQ ID NO: 22 through SEQ ID NO:39 inclusive.

50. The method of claim 43, wherein the nucleic acid from the tumor or normal tissue is labeled with a detectable label.

51. The method of claim 50, wherein the detectable label is a fluorescent label.

52. The method of claim 51 wherein
(a) nucleic acids from the tumor and the tissue are labeled with a fluorescent label prior to the hybridization; and
(b) the hybridization is detected as a fluorescent signal bound to the probe.

53. The method of claim 43, wherein
(a) the probes are immobilized to a solid surface in a microarray of pixels; and
(b) the tumor tissue or normal kidney tissue samples are spotted onto the immobilized probe pixels.

54. The method of claim 53 wherein
(a) the immobilized probes are arranged as pixels in rows; and
(b) the tumor tissue or normal kidney tissue samples are spotted column-wise onto the probe pixels.

55. A method for early diagnosis in a subject of (i) a CC-RCC tumor prior to physical or radiological evidence of the tumor, or (ii) recurrence of a CC-RCC tumor after excision or other treatment of a CC-RCC primary tumor, the method comprising:
(a) selecting a protein product of at least one gene, the expression of which is up-regulated in a majority of CC-RCC patients, which protein is a secreted protein or is expressed on cell surfaces in a tissue that is readily accessible for assay; and
(b) determining the presence or measuring the quantity of the protein product in a body fluid or a tissue or cell sample from the subject,
wherein, an increased level of the protein product compared to
(i) the level of the protein in a normal subject's body fluid, tissue or cells, or
(ii) another reference normal value for the protein level,
is indicative of the presence of said CC-RCC tumor in the subject.

56. The method of claim 55 wherein the gene is one that hybridizes with one or more of:
(a) SEQ ID NO:40-SEQ ID NO:68; or
(b) SEQ ID NO:140-SEQ ID NO:230.

57. The method of claim 56 wherein the gene is one that hybridizes with one or more of SEQ ID NO:40-SEQ ID NO:68

58. An array of immobilized nucleic acid probes useful in a method of predicting whether CC-RCC in a subject is of a non-aggressive or aggressive type, or in a method for early diagnosis of a primary or recurring CC-RCC tumor, comprising:
(i) a first set of probes of any one or more of SEQ ID NO:1-SEQ ID NO:39 inclusive, SEQ ID NO:139 or SEQ ID NO:332-SEQ ID NO:497, inclusive, which first set probes are complementary to nucleic acid sequences of genes expressed differentially in aggressive as compared to non-aggressive types of CC-RCC; or
(ii) a second set of probes of any one or more of SEQ ID NO:40-SEQ ID NO:68 or SEQ ID NO:140-SEQ ID NO:230, inclusive, which second set probes are complementary to nucleic acid sequences of genes expressed differentially in a majority of CC-RCC patients compared to normal subjects or to another reference normal value,
which nucleic acid sequences hybridize to the probes under high stringency conditions.

59. The array of claim 58 which is a microarray wherein, the probes are immobilized in predetermined order such that a row of pixels corresponds to replicates of one distinct probe from the set.

60. The microarray of claim 59, wherein the set of probes comprises at least 10 cDNA probes have the sequence SEQ ID NO: 1-SEQ ID NO:10.

61. The microarray of claim 59, wherein the set of probes comprises at least 39 cDNA probes having the sequence SEQ ID NO: 1-SEQ ID NO:39.

62. The microarray of claim 59, wherein the set of probes comprises at least 206 cDNA probes having the sequence SEQ ID NO:1-SEQ ID NO:39, SEQ ID NO:139 and SEQ ID NO:332-SEQ ID NO:497.

63. The microarray of claim 59, wherein the probes comprise nucleotides having at least one modified phosphate backbone selected from a phosphorothioate, a phosphoridothioate, a phosphoramidothioate, a phosphoramidate, a phosphordiimidate, a methylsphosphonate, an alkyl phosphotriester, 3′-aminopropyl, a formacetal, or an analogue thereof.

64. The microarray of claim 59, wherein each probes comprises at least 15 nucleotides.

65. The microarray of claim 59, further comprising one or more nucleic acid samples representing expressed genes, each sample from an individual subject's tumor or normal tissue, each sample spotted column-wise on the pixels of the microarray probes.

66. The microarray of claim 62, which has further been subjected to nucleic acid hybridization under high stringency conditions such that the nucleic acid samples are hybridized with the immobilized probes on which the samples have been spotted.

67. A composition comprising a combination of two or more isolated oligonucleotide or polynucleotide probes each of which hybridizes with part or all of a coding sequence that is differentially expressed in
(i) CC-RCC tumors compared to normal kidney tissue, and/or
(ii) an aggressive type of CC-RCC compared to a non-aggressive type of CC-RCC.

68. The composition of claim 67 wherein the probes are immobilized to a solid surface in predetermined order such that a row of probe pixels corresponds to replicates of one distinct probe from the combination.

69. The composition of claim 67 comprising a combination of at least 2 of the probes.

70. The composition of claim 69 comprising a combination of at least 10 of the probes.

71. The composition of claim 70 comprising a combination of at least 39 of the probes.

72. The composition of claim 71 comprising a combination of at least 99 of the probes.

73. The composition of claim 72 comprising a combination of at least 206of the probes.

74. The composition of claim 73 comprising a combination of at least 291 of the probes.

75. The composition of claim 67, wherein the coding sequence is up-regulated in the aggressive CC-RCC compared to normal kidney tissue.

76. The composition of claim 67, wherein the coding sequence is down-regulated in the aggressive CC-RCC compared to normal kidney tissue.

77. The composition of claim 67, wherein the coding sequence is up-regulated in the non-aggressive CC-RCC compared to normal kidney tissue.

78. The composition of claim 67, wherein the coding sequence is down-regulated in the non-aggressive CC-RCC compared to normal kidney tissue.

79. A kit for evaluating expression of nucleic acids said kit being compartmentalized to receive in close confinement therein one or more containers, said kit comprising:
(a) an array according to claim 58;
(b) reagents that facilitate either one or both of (i) hybridization of the nucleic acid to the immobilized probes and (ii) detection of said hybridization; and
(c) optionally, a computer readable storage medium comprising logic which enables a processor to read data representing detection of hybridization.

80. A kit for evaluating expression of nucleic acids said kit being compartmentalized to receive in close confinement therein one or more containers, said kit comprising:
(a) a composition according to claim 67;
(b) reagents that facilitate either one or both of (i) hybridization of the nucleic acid to the immobilized probes and (ii) detection of said hybridization; and
(c) optionally, a computer readable storage medium comprising logic which enables a processor to read data representing detection of hybridization.

81. The kit of claim 79 wherein the array is a microarray on a solid surface and the probes are immobilized to the surface in predetermined order such that a row of probe pixels corresponds to replicates of one distinct probe from the first and/or the second probe set.

82. The kit of claim 79 wherein the detection employs fluorescence.

83. A kit for evaluating expression of nucleic acids said kit being compartmentalized to receive in close confinement therein one or more containers, said kit comprising:
(a) the array of claim 58;
(b) means for carrying out hybridization of the nucleic acid to the probes; and
(c) means for reading hybridization data.

84. A kit for evaluating expression of nucleic acids said kit being compartmentalized to receive in close confinement therein one or more containers, said kit comprising:
(a) the composition of claim 67;
(b) means for carrying out hybridization of the nucleic acid to the probes; and
(c) means for reading hybridization data.

85. The kit of claim 83 wherein the array is a microarray on a solid surface and the probes are immobilized to the surface in predetermined order such that a row of probe pixels corresponds to replicates of one distinct probe from the first and/or the second probe set.

86. The kit of claim 83, wherein the hybridization data being read is in the form of fluorescence data.
